(19) United States
(12) Patent Application Publication
(10) Pub. No.: US 2005/0261243 A1
(43) Pub. Date: Nov. 24, 2005

(54) Antiprostaglandins for the treatment of ocular pathologies

(76) Inventor: Gholam Ali Peyman, New Orleans, LA (US)

Correspondence Address: FROMMER LAWRENCE & HAUG, 745 FIFTH AVENUE- 10TH FL., NEW YORK, NY, 10151, US

(21) Appl. No.: 11/113,450

(22) Filed: Apr. 21, 2005

Related U.S. Application Data
(63) Continuation-in-part of application No. 10/828,982, filed on Apr. 21, 2004, now abandoned.
(63) Continuation-in-part of application No. 10/935,850, filed on Sep. 8, 2004, now abandoned.
(63) Continuation-in-part of application No. 10/936,303, filed on Sep. 8, 2004, now abandoned.

(30) Foreign Application Priority Data
Dec. 3, 2004 (AU) 2004906932

(51) Int. Cl. A61K 31/192; A61K 31/727

(52) U.S. Cl. 514/56; 514/569

(57) ABSTRACT

Formulations and methods useful to treat ocular neovascularization (new blood vessel growth in the cornea, retina, conjunctiva, and/or choroid) are disclosed. According to the invention there is provided a formulation suitable for the treatment of ocular neovascularization that may comprise flurbiprofen in a concentration and dose suitable for treating ocular neovascularization, characterized in that said flurbiprofen may be at a substantially neutral pH in a pharmaceutically acceptable form suitable for delivery to the eye.

### INCORPORATION BY REFERENCE

This application is a continuation-in-part of U.S. patent application Ser. No. 10/828,982 filed 21 Apr. 2004, U.S. patent application Ser. No. 10/935,850 filed 8 Sep. 2004, U.S. patent application Ser. No. 10/936,303 filed 8 Sep. 2004, and Australian Provisional Patent Application No. 2004906932 filed 3 Dec. 2004.

The foregoing applications, and all documents cited therein, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

Disclosed herein are formulations for the treatment of ocular neovascularization as well as treatment regimens that limit, reduce, slow the rate of, or prevent ocular neovascularization, and/or that cause regression of existing new blood vessels, in a patient with an ocular pathology.

### BACKGROUND

Ocular neovascularization is the pathologic in-growth of blood vessels in the cornea, retina, or choroid. Blood vessel growth or formation can be due to diverse events and may lead to sight threatening conditions and even blindness due to bleeding and subsequent scarring, fibrosis, etc. Causes of blood vessel growth or formation include hypoxia (e.g., in diabetes), inflammatory responses (e.g., keratitis due to autoimmune disease), microbial infection (e.g., keratitis, blepharitis), physical insult (e.g., improper use of contact lenses), chemical insult (e.g., toxins), pharmacologic agents, or other factors (e.g., graft rejection). More specifically, an inflammatory response may follow corneal transplant. Ocular microbial infections include but are not limited to trachoma, viral interstitial keratitis, and keratoconjunctivitis. Physical insult (such as corneal insult) may be due to contact with acidic or alkaline solutions, trauma, improper hygiene and/or compliance with contact lens use, such as extended wear lenses, or chemical agents such as silver nitrate. Other factors leading to ocular neovascularization include mechanical irritation of the limbal sulcus, corneal hypoxia, epithelial cell erosion or hypertrophy. In dry eye disease (conjunctiva sicca), the dehydrated conditions cause sloughing off of the epithelium, resulting in new vessel formation.

One form of ocular neovascularization that is a major public health problem is neovascularization of the cornea, which is a major cause of ocular morbidity. In the USA alone, corneal neovascularization (CoNV) is estimated to occur in 1.4 million patients (4% of the U.S. population) each year. CoNV may occur in a wide range of diseases affecting the cornea. For example, it may result from inflammatory conditions (such as chemical burns), immunologically mediated conditions (such as herpes simplex keratitis), allograft reactions, or extended wear contact lenses. These insults can lead to invasion of capillaries from the limbal plexus, resulting in CoNV which may lead to decreased visual acuity secondary to stromal edema, lipidic deposits, causal keratitis, and scarring.

The pathogenesis of CoNV has not yet been fully clarified in terms of identification and significance of angiogenic and anti-angiogenic factors. What is known is that corneal avascularity requires a balance between angiogenic and anti-angiogenic molecules. If this homeostasis is disrupted, it may result in neovascularization. More particularly, CoNV occurs when there is up-regulation of angiogenic factors or down regulation of anti-angiogenic molecules. Several angiogenic and anti-angiogenic molecules have been isolated from the cornea.

Numerous substances accelerate new vessel formation such as various growth factors (fibroblast growth factor, transforming growth factor, tumour necrosis factor, etc.), prostaglandins and interleukins. Various compounds have been identified as inhibitors in experimental and clinical CoNV including steroids, nonsteroidal anti-inflammatory drugs, cyclosporin A, methotrexate, FK506, thalidomide and other anti-angiogenic factors.

Methods of treating ocular neovascularization have met with limited success. Although there is no clear consensus, methods include treatment of the underlying condition, if possible; topical corticosteroid application for gross and active neovascularization; diathermy of large feeding vessels and corneal laser photocoagulation for treatment of superficial neovascularization of the cornea with infiltration of granulation tissue (pannus); and even timbal grafting for severe chemical injuries and limbal epithelium loss.

Topical corticosteroids have been the mainstay of prevention and treatment for CoNV, but they are not always effective and sometimes may be associated with serious complications such as cataract, ocular hypertension, glaucoma, and infections. Recognition of the potential side effects of corticosteroids in their use for angiogenesis has led to a search for other natural or synthetic angiogenesis inhibitors. Although corticosteroids have been known for a long time to be useful agents in prevention of CoNV in various clinical and experimental circumstances, there has not been enough research related with usage in combination with other drugs.

Other methods and formulations which reduce or prevent ocular neovascularization, and which may treat an ocular pathology, are desirable.

Citation or identification of any document in this application is not an admission that such document is available as prior art to the present invention.

### SUMMARY OF THE INVENTION

Formulations and methods useful to treat ocular neovascularization (new blood vessel growth in the cornea, retina, conjunctiva, and/or choroid) are disclosed. According to the invention there is provided a formulation suitable for the treatment of ocular neovascularization that may comprise antiprostaglandins in a concentration and dose suitable for treating ocular neovascularization, characterized in that said compound is in a pharmaceutically acceptable form suitable for delivery to the eye. The use of drugs such as steroids in the treatment of ocular neovascularization ailments can increase intraocular pressure (glaucoma). Accordingly, a formulation according to the present invention is at least beneficial for patients with glaucoma or at risk for glaucoma, and for patients after glaucoma filtering surgery.

Desirably, there is provided a formulation suitable for the treatment of ocular neovascularization that may comprise antiprostaglandins in a concentration and dose suitable for treating ocular neovascularization, characterized in that said compound is at a substantially neutral pH in a pharmaceutically acceptable form suitable for delivery to the eye.

According to the invention pharmaceutically acceptable formulations are desirably prepared for topical ocular application, ocular injection, or ocular implantation, and may be contained in liposomes or slow release capsules.

Preferably the formulation that may comprise flurbiprofen in a concentration and dose suitable for treating ocular neovascularization (for example at a concentration from about 0.001%^{w/v} to about 0.5%^{w/v} of flurbiprofen), characterized in that said compound may be at a substantially neutral pH in a pharmaceutically acceptable form suitable for delivery to the eye.

In a more preferable form of the invention there is provided a pharmaceutically acceptable formulation suitable for treating ocular neovascularization that may comprise: (a) an antiprostaglandin (such as flurbiprofen) in a concentration from about 0.001%^{w/v} to about 0.5% ^{w/v}, characterized in that said compound is at a substantially neutral pH in a pharmaceutically acceptable form suitable for delivery to the eye; and may further comprise (b) at least another compound in a concentration and dose sufficient to reduce ocular neovascularization selected from the group consisting of: a tetracycline, a steroid, an antimicrobial, heparin, and/or a metalloproteinase inhibitor. More preferably, the formulation may include a plurality of compounds in a concentration and dose suitable for reducing ocular neovascularization selected from the group consisting of: a tetracycline, a steroid, an antimicrobial, heparin, and/or a metalloproteinase inhibitor.

In a form of this embodiment, the invention is a formulation that may comprise: an antiprostaglandin in a concentration from about 0.001%^{w/v} to about 0.5%^{w/v}, characterized in that said compound may be at a substantially neutral pH in a pharmaceutically acceptable form suitable for delivery to the eye and a tetracycline derivative (such as doxycycline, demeclocycline, minocycline, oxytetracycline, lymecycline, or a chemically modified tetracycline) at a concentration from about 0.01 pg/ml to about 30 mg/ml.

In a second form of this embodiment, the invention is a formulation comprising: a an antiprostaglandin in a concentration from about 0.001%^{w/v} to about 0.5%^{w/v}, characterized in that said compound is at a substantially neutral pH in a pharmaceutically acceptable form suitable for delivery to the eye and a steroid in a concentration and dose sufficient to reduce ocular neovascularization, for example at a dose of about 1 mg/ml to about 8 mg/ml.

In a third form of this embodiment the invention is a formulation that may comprise: an antiprostaglandin in a concentration from about 0.001%^{w/v} to about 0.5%^{w/v}, characterized in that said compound may be at a substantially neutral pH in a pharmaceutically acceptable form suitable for delivery to the eye and a low molecular weight heparin in a concentration and dose sufficient to reduce ocular neovascularization, for example in a concentration from about 0.01 pg/ml to about 100 mg/ml.

In a fourth form of this embodiment the invention is a formulation that may comprise: an antiprostaglandin in a concentration from about 0.001%^{w/v} to about 0.5%^{w/v}, characterized in that said compound may be at a substantially neutral pH in a pharmaceutically acceptable form suitable for delivery to the eye and a antimicrobial in a concentration and dose sufficient to reduce ocular neovascularization.

In a fifth form of this embodiment the invention is a formulation that may comprise: an antiprostaglandin in a concentration from about 0.001%^{w/v} to about 0.5%^{w/v}, characterized in that said compound may be at a substantially neutral pH in a pharmaceutically acceptable form suitable for delivery to the eye and an inhibitor of a metalloproteinase in a concentration and dose sufficient to reduce ocular neovascularization.

In a sixth form of this embodiment the invention is a formulation that may comprise: an antiprostaglandin in a concentration from about 0.001%^{w/v} to about 0.5%^{w/v}, characterized in that said compound may be at a substantially neutral pH in a pharmaceutically acceptable form suitable for delivery to the eye and a steroid in a concentration and dose sufficient to reduce ocular neovascularization and doxycycline in a concentration and dose sufficient to reduce ocular neovascularization.

In another form the invention resides in a method for reducing ocular neovascularization said method comprising the steps of administering to the eye of a patient an antiprostaglandin in a concentration from about 0.001%^{w/v} to about 0.5%^{w/v}, characterized in that said compound may be at a substantially neutral pH in a pharmaceutically acceptable form suitable for delivery to the eye. Administration may be by topical, subconjunctival or intraocular routes or via ocular implants. The method may reduce neovascularization in the anterior and/or posterior portions of the eye, or in the cornea, retina, choroid, etc.

Other objects, features, and advantages of the instant invention, in its details as seen from the above, and from the following description when considered in light of the appended claims.

### BRIEF DESCRIPTION OF THE DRAWINGS

Comprehension of the invention is facilitated by reading the following detailed description, in conjunction with the annexed drawings.

FIG. 1A is a photograph of a rat eye to which flurbiprofen and low molecular weight heparin were administered.

FIG. 1B is a photograph of a rat eye to which flurbiprofen and doxycycline were administered.

FIG. 1C is a photograph of a rat eye to which doxycycline and low molecular weight heparin were administered.

FIG. 1D is a photograph of a rat eye to which a balanced salt solution was administered.

FIG. 2 is a graph showing the effect of various agents, on percentage of the cornea occupied by blood vessels.

FIG. 3A is a photograph of a histological preparation of a rat eye to which flurbiprofen and doxycycline were administered.

FIG. 3B is a photograph of a histological preparation of a rat eye to which a balanced salt solution was administered.

FIG. 4 is a bar chart demonstrating the percentage of cornea occupied by blood vessels in each group (LMWH: low molecular weight heparin, ASC: ascomycin, Flur: flurbiprofen, DOX: doxycycline, and TA: triamcinolone). Lines under the x-axis connect groups that are not significantly different from each other (p>0.5).

FIG. 5A is a slit lamp photograph of the cornea seven days after induction of corneal burn in control eyes receiving normal saline.

FIG. 5B is a digitally enhanced version of FIG. 5A, accentuating the blood vessels.

FIG. 6A is a digitally enhanced slit lamp photograph of the cornea seven days after induction of corneal burn in eyes treated with flurbiprofen (neovascularization is quite prominent in this group).

FIG. 6B is a digitally enhanced slit lamp photograph of the cornea seven days after induction of corneal burn in eyes treated with doxycycline (neovascularization is less prominent than in control group).

FIG. 6C is a digitally enhanced slit lamp photograph of the cornea seven days after induction of corneal burn in eyes treated with triamcinolone acetonide (arrows circumscribe the relatively small neovascular area).

FIG. 7A is a photograph of a histopathology preparation of the corneal burn in a control eye treated with normal saline, showing corneal scar (large arrow) and new vessels (small arrows) in the corneal stroma. H&E 100X.

FIG. 7B is a photograph of a histopathology preparation of the corneal burn in an eye treated with triamcinolone acetonide (double arrows point to avascular stroma). Note extensive neovascularization of the corneal stroma in FIG. 7A compared to FIG. 7B. H&E 100X.

### DESCRIPTION OF THE INVENTION

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

The file of this patent contains at least one drawing executed in color. Copies of this patent with color drawing(s) will be provided by the Patent and Trademark Office upon request and payment of the necessary fee.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the description of the invention and apply throughout. Unless otherwise defined, all other scientific and technical terms used herein have the same meaning as commonly understood to one of ordinary skill in the art to which the invention belongs.

Disclosed herein are formulations for the treatment of ocular neovascularization as well as treatment regimens that limit, reduce, slow the rate of, or prevent ocular neovascularization, and/or that cause regression of existing or new blood vessels, generally referred to as reduced neovascularization, although the term encompasses any degree of inhibition by any method and also encompasses any degree of regression of existing vessels.

Ocular neovascularizations can be superficial or deep and may lead to loss of optical transparency through stromal hemorrhage, scarring, lipid deposition, etc. Neovascularization may occur in any area of the eye, such as the cornea, retina, conjunctiva, or choroid. The presence of new vessels may result in an increased intraocular pressure, termed neovascular glaucoma or ocular hypertension. The new vessels may lead to hemorrhage and fibrosis, and result in structural damage to the eye with subsequent decreased visual acuity. For example, corneal burns result in the formation of new vessels that can decrease vision as they infiltrate and penetrate the cornea. In corneal transplants, new blood vessels from the limbus penetrate the cornea and may result in rejection of the engrafted tissues. Thus, control or prevention of new vessels to any extent is desirable, although greater inhibition is more desirable and total inhibition of new vessels is most desirable.

As used herein the phrase “ocular neovascularization” refers to any ocular disorder or pathological condition of the eye, i.e. ocular disease, which is caused by vessel growth or proliferation as a component to the disease state. Such ocular diseases can include, inter alia, but are not limited to: ocular neovascularization; retinal diseases (such as diabetic retinopathy, sickle cell retinopathy, retinopathy of prematurity, macular degeneration (eg early onset macular degeneration, neovascular macular degeneration, age-related macular degeneration)); iritis; rubeosis iritis; inflammatory diseases; anterior and posterior uveitis including chronic uveitis; neoplasms (retinoblastoma, pseudoglioma); Fuchs' heterochromic iridocyclitis; neovascular glaucoma; corneal neovascularization (inflammatory, transplantation); ischemic retinopathies; sequelae vascular diseases (retinal ischemia, choroidal vascular insufficiency, choroidal thrombosis, carotid artery ischemia); choroidal neovascularization; pterygium; neovascularization of the optic nerve; neovascularization due to penetration of the eye or contusive ocular injury and exudative retinopathies like myopic retinopathies, cystoid macular edema arising from various aetiologies, exudative macular degeneration, diabetic macular edema, central vein occlusion, branch vein occlusion; retinitis of prematurity, cyclitis, sickle cell retinopathy and macular edema arising from laser treatment of the retina or as a post-operative treatment, e.g. after corneal transplant or ocular surgery including corneal surgery (e.g., LASIK® surgery, photorefractive keratectomy (PRK), or other corneal procedures. The inventive methods and formulations may desirably inhibit ocular neovascularization that occurs from any event, for example, due to ocular disease, hypoxia, trauma, physical or chemical insult, etc.

In various embodiments, doses and formulations of the inventive formulation are administered to a patient in addition to, or as treatments for, an ocular neovascularization pathology.

According to the invention there is provided a formulation suitable for the treatment of ocular neovascularization comprising an antiprostaglandin in a concentration and dose suitable for treating ocular neovascularization, characterized in that said compound is in a pharmaceutically acceptable form suitable for delivery to the eye. According to the invention the formulations are preferably in pharmaceutically acceptable formulations for topical ocular application, ocular injection, or ocular implantation, and may be contained in liposomes or slow release capsules.

It will be appreciated that the antiprostaglandin employed in the invention need only be in a form where it can be administered to or applied to the eye or its surrounding tissue. Thus, the antiprostaglandin may be prepared in an acidic or basic environment and/or may even be provided in a final form suitable for administration in this form. Preferably, however, the antiprostaglandin is prepared in a form suitable for administrations to the ocular environment. More preferably, it is prepared in a manner which results in the final formulation having some physiologically compatibility with the eye. For example, if the formulation is to be injected into the eye the formulation should be physiologically suitable for ocular insertion. Likewise if the formulation is prepared for topical administration then it may be in a form that is not necessarily physiologically compatible with the ocular environment, but by the time the compound(s) reaches its site of action it is so compatible.

Desirably there is provided a formulation suitable for the treatment of ocular neovascularization comprising an antiprostaglandin in a concentration and dose suitable for treating ocular neovascularization, characterized in that said compound is at a substantially neutral pH in a pharmaceutically acceptable form suitable for delivery to the eye.

As used herein the phrase “substantially neutral pH” refers to a pH that is between about 5 and about 9 and would include pHs such as 5.5, 6, 6.5, 7, 7.5, 8, and 8.5 and variations in such pHs. Where the phrase is used in conjunction with a formulation that is to be injected into the ocular environment, the phrase will have the additional limitation that the final formulation for administration will be at or about a pH that is substantially compatible with the ocular environment.

The concentration of antiprostaglandin employed in the formulation ranges from about 0.001%^{w/v} to about 0.5%^{w/v}. Preferably the antiprostaglandin is administered in a substantially non-toxic amount or concentration, which may depend on the route of administration, the compound employed and a host of patient related factors.

Antiprostaglandins, also termed prostaglandin antagonists, may be administered in a concentration sufficient to result in a prostaglandin-inhibitory effect. As one example, antiprostaglandins such as flurbiprofen may be administered at a concentration in the range of about 0.001%^{w/v} to about 0.5%^{w/v}. As an example, 0.03% OCUFEN® (flurbiprofen sodium 0.03% (Allergan); sodium (±)-2-(2-fluoro-4-biphenylyl)-propionate dihydrate) may be administered at a concentration ranging from about 0.003%^{w/w} to about 0.3%^{w/w}. Antiprostaglandins other than flurbiprofen may be included. The antiprostaglandins maybe administered at the doses and by the methods previously described, and include indomethacin, ketorolac, tromethamine 0.5% (±)-5-benzoyl-2,3-dihydro-1H-pyrrolizine-1-carboxylic acid, compound with 2-amino-2-(hydroxymethyl)-1,3-propanediol (1:1) (ACULAR® Allegan, Irvine Calif.), meclofenamate, fluorbiprofen, and compounds in the pyrrolo-pyrrole group of non-steroidal anti-inflammatory drugs (NSAIDs). For example, ACULAR® may be administered at a concentration ranging from about 0.003%^{w/w} to about 0.3%^{w/w}. In one embodiment, the concentration of ACULAR® is about 0.03%^{w/w}.

In another embodiment the invention resides in an ocular pharmaceutically acceptable formulation (that is, containing buffers and excipients as known to one skilled in the art) which comprises: (a) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization; and (b) at least a compound in a concentration and dose sufficient to reduce ocular neovascularization wherein the compounds are selected from the group consisting of: a tetracycline or a derivative thereof (including CMTs which inhibit MMP activity), a steroid, an antimicrobial, heparin, and/or a metalloproteinase inhibitor.

In one form, formulations of the invention comprise an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and a tetracycline or a derivative thereof (including CMTs which inhibit MMP activity) in a concentration from about 0.01 pg/ml to about 30 mg/ml.

As used herein a tetracycline or a derivative thereof including CMTs which inhibit MMP activity will include, inter alia: doxycycline, demeclocycline, minocycline, oxytetracycline, lymecycline, or a chemically modified tetracycline. Chemically modified tetracyclines (CMT) include demeclocycline, minocyciine, oxytetracycline and like compounds that inhibit the synthesis of MMP-2, MMP-8 and MMP-9. These include CMT such as CMT-315, CMT-3, CMT-8, and CMT-308; 6-demethyl-6-deoxy-4-dedimethylaminotetracylcine (COL-3), and others, for example, as described by Liu et al., A Chemically Modified Tetracycline (CMT-3) Is a New Antifungal Agent in Antimicrobial Agents Chemother. 2002 May; 46:1447; Seftor et al., Targeting the Tumor Microenvironment with Chemically Modified Tetracyclines: Inhibition of Laminin 5γ2 Chain Promigratory Fragments and Vasculogenic Mimicry 2002 November; 1: 1173, which are expressly incorporated by reference herein.

Tetracyclines exert their biological effects independent of their antimicrobial activity. That is, they inhibit MMPs and can prevent pathogenic tissue destruction. Furthermore, recent studies have suggested that tetracyclines and inhibitors of metalloproteinases suppress tumor progression, bone resorption, and angiogenesis and may have anti-inflammatory properties. Thus, a possible mechanism for the beneficial effect of tetracyclines and like compounds in reducing vessel growth and proliferation in the ocular region is via inhibition of metalloproteinases, which are zinc-dependent proteinase enzymes associated with the tumorigenic process. Selective inhibition of such metalloproteinase by the inventive formulations and methods described herein is believed to inhibit reactions leading to ocular neovascularization. Such metalloproteinase inhibitors are also included in the invention.

In a highly preferred form of the invention, doxycycline is the tetracycline derivative employed in the formulation. Doxycycline (4-(dimethylamino)-1,4,4a,5,5a,6,11,12a-octahydro3,5,10,12,12a-pentahydroxy-6-methyl-1,11-dioxo-2-naphthacenecarboxamide monohydrate, C₂₂H₂₄N₂O₈.H₂O) is a broad spectrum antibiotic in the class of tetracycline antibiotics. It is commercially available.

According to this form of the invention the formulation comprises doxycycline in an amount sufficient to reduce ocular neovascularization together with excipients for topical, subconjunctival, or intraocular administration. For example the formulation might contain 2% doxycycline at a substantially neutral pH.

The concentration of doxycycline employed in this form of the invention will range from 0.01 μg/ml to about 30 mg/ml. More specifically, doxycycline concentrations will range from about 0.05 mg/ml to about 1 mg/ml. Alternatively doxycycline concentrations will range from about 0.05 mg/ml to about 10 mg/ml. Yet again doxycycline concentrations can range from about 1 mg/ml to about 20 mg/ml. These doses are substantially non-toxic to the patient. Besides its anti-angiogenic effect, doxycycline could reduce the incidence of endophthalmitis, which occurs in about 0.5% of eyes in which an intravitreal steroid is administered.

In a highly preferred form the following formulation may be used: a 1:1 combination of about 0.03%^{w/v} flurbiprofen and about 20 mg/ml doxycycline.

In second form formulations of the invention comprise an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and a steroid at a concentration from about 0.1 mg/ml to about 40 mg/ml.

Steroids are usually administered for ocular pathologies such as uveitis, diabetic retinopathy, idiopathic juxtafoveal telangiectasias, macular edema secondary to diabetes mellitus, central retinal vein occlusion, pseudophakia, during photodynamic therapy for age related macular degeneration, etc., and for intraoperative visualization of the posterior hyaloid, which also desirably inhibit ocular neovascularization. An undesirable and serious side effect of ocular steroid therapy is increased intraocular pressure, termed glaucoma or ocular hypertension. For patients with glaucoma or predisposed to glaucoma, steroid therapy presents a risk for unacceptably high intraocular pressure, such that surgery may be required to lower the intraocular pressure. Such risks and benefits must be balanced in determining whether to treat the patient with triamcinolone or other steroids. The formulations and methods to predict patients at risk for glaucoma from steroid therapy, disclosed in co-pending U.S. patent application Ser. No. 10/787,580, which is expressly incorporated by reference herein in its entirety.

Steroids for ocular administration include, but are not limited to, triamcinolone (Aristocort®; Kenalog®), betamethasone (Celestone®), budesonide, cortisone, dexamethasone (Decadron-LA®; Decadron® phosphate; Maxidex® and Tobradex® (Alcon)), hydrocortisone, methylprednisolone (Depo-Medrol®, Solu-Medrol®), prednisolone (prednisolone acetate, e.g., Pred Forte® (Allergan); Econopred and Econopred Plus® (Alcon); AK-Tate® (Akorn); Pred Mild® (Allergan); prednisone sodium phosphate (Inflamase Mild and Inflamase Forte® (Ciba); Metreton® (Schering); AK-Pred® (Akorn)), fluorometholone (fluorometholone acetate (Flarex® (Alcon); Eflone®), fluorometholone alcohol (FML® and FML-Mild®, (Allergan); FluorOP®)), rimexolone (Vexol® (Alcon)), medrysone alcohol (HMSO (Allergan)); lotoprednol etabonate (Lotemax® and Alre)® (Bausch & Lomb), 11-desoxcortisol, and anecortave acetate (Alcon)). It will be appreciated that the above lists are representative only and are not exclusive.

In a highly preferred form of the invention the steroid used in the formulation is a 11-substituted-16α,17α-substituted methylenedioxy steroid selected from the compounds disclosed in U.S. Pat. No. 5,770,589 to Billson and Penfold (“US '589”), which was filed as U.S. application Ser. No. 08/586,750, and is incorporated herein in its entirety by reference. Alternatively the compound is a steroid disclosed in Fried et al. (1958) J. Am. Chem. Soc. 80, 2338 (1958); U.S. Pat. No. 2,990,401; U.S. Pat. No. 3,048,581 or U.S. Pat. No. 3,035,050 each of which is expressly herein incorporated by reference. Collectively these publications also provide methods for the manufacture of such compounds and are also incorporated for the purposes of disclosing such methods. Desirably the steroid used in the method is triamcinolone acetonide.

The steroid concentration in the inventive formulation ranges from about 0.1 mg/ml to about 40 mg/ml. More preferably the steroid concentration ranges from about 1 mg/ml to about 20 mg/ml. Alternatively, steroid concentrations range from about 20 mg/ml to about 30 mg/ml or they can range from about 20 mg/ml to about 40 mg/ml.

The steroid concentration used with a particular formulation will depend upon the particular steroid that is used. For example, triamcinolone acetonide (9α-fluoro-1 1 13,16a,17,21 tetra hydroxy-pregna-1,4-diene-3,20-dione cyclic 16,17-acetal with acetone (C₂₄H₃₁FO₆)) Kenacort®, Kenalog® (Bristol-Myers Squibb, Princeton N.J.) may be administered at a therapeutic dose in the range of about 4 mg to about 8 mg, for example, by intravitreous injection. In comparison, anecortave acetate, a steroid with less potential to cause an increase in intraocular pressure than triamcinolone (but administered as a posterior juxtascleral depot rather than in the eye), may be administered at dose of about 0.5 mg/ml to about 30 mg/ml.

In a highly preferred form the following formulation may be used: a 1:1 combination of about 0.03%^{w/v} flurbiprofen and about 4 mg/ml steroid.

In a third form, formulations of the invention comprise an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and heparin or low molecular weight heparin in a concentration from about 0.01 pg/ml to about 100 mg/ml.

Heparin is a heterogeneous group of straight-chain anionic mucopolysaccharides, termed glycosaminoglycans, having anticoagulant activity. The primary sugars are a-L-iduronic acid 2-sulfate, 2-deoxy-2-sulfamino-α-D-glucose 6-sulfate, (3-D-glucuronic acid, 2-acetamido-2-deoxy-a-D-glucose, and α-L-iduronic acid. These sugars are present in different amounts and are joined by glycosidic linkages, forming polymers of varying sizes. Heparin is strongly acidic because of its content of covalently linked sulfate and carboxylic acid groups. In heparin sodium, the acidic protons of the sulfates are partially replaced by sodium ions. In one embodiment of the invention, low molecular weight heparin is used. Low molecular weight heparin is derived from standard heparin through either chemical or enzymatic depolymerization, and is commercially available. Standard heparin has a molecular weight of about 5,000 daltons to about 30,000 daltons, while low molecular weight heparin has a molecular weight of about 1,000 daltons to about 10,000 daltons. Compared to standard heparin, low molecular weight heparin binds less strongly to protein, has enhanced bioavailability, interacts less with platelets and yields a predictable dose response and dose-dependent plasma levels, and produces less bleeding for a given antithrombotic effect. Low molecular weight heparin may be heparin sulfate, a lower-sulfated, higher-acetylated form of heparin. All of these are commercially available (e.g., Sigma Aldrich, St. Louis Mo.).

A possible mechanism for the beneficial effect of heparin or low molecular weight heparin in reducing vessel growth and proliferation is its polyanionic structure, which readily binds to polycationic angiogenic factors. Angiogenic factors with heparin bound to them have reduced biological activity, and therefore do not promote new vessel growth. In vivo, heparin sulfates are bound to the extracellular matrix (ECM) and endothelial cell surfaces. Heparin sulfate in the ECM may have a role in storing active growth factors that can be released when needed to exert immediate effects. Soluble heparins compete with heparin sulfates on the ECM for growth factors and proteins, and may consequently cause their release. Unfractionated heparin (UFH) may cause an increase in the plasma level of growth factors. Unlike UFH, which may promote angiogenesis, low molecular weight heparin may hinder the binding of growth factors to their high affinity receptors as a result of its smaller size. Low molecular weight heparin may affect the injured neovascular cornea by binding angiogenic factors that have been released from the ECM, as well as competitively (antagonistically) binding to angiogenic receptors.

In one embodiment, the concentration of heparin or low molecular weight heparin ranges from about 0.01 pg/ml to about 30 mg/ml. Alternatively, heparin or low molecular weight heparin may be administered in a concentration ranging from about 1 mg/ml to about 10 mg/ml. In a more preferred form of the invention, the concentration of heparin or low molecular weight heparin ranges from about 0.5 mg/ml to about 15 mg/ml to 20 mg/ml (for example, administration of 0.1 ml of a 100 mg/ml formulation of low molecular weight heparin). In various embodiments, the concentration may be about 0.5 mg/ml to about 2.5 mg/ml, about 1 mg/ml to about 5 mg/ml, about 5 mg/ml to about 10 mg/ml, or about 5 mg/ml to about 30 mg/ml. Any concentration within these ranges may be used.

In a highly preferred form the following formulation may be used: a 1:1 combination of about 0.03%^{w/v} flurbiprofen and about 10 mg/ml heparin or low molecular weight heparin.

In a fourth form, formulations of the invention comprise an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and a antimicrobial, for example a macrolide antibiotic, in a concentration from about 20 μg/ml to about 200 μg/ml (about 0.002%^{w/v} to about 0.02%^{w/v}).

A possible mechanism for the beneficial effect of macrolide antibiotics are their anti-inflammatory effect.

Macrolide antibiotics that can be added to the inventive formulation include, inter alia: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide. Other antibiotics include, but are not limited to, aminoglycosides (e.g., streptomycin, amikacin, gentamicin, tobramycin), cephalosporins (e.g., beta lactams including penicillin), tetracyclines, acyclorvir, amantadine, polymyxin B, amphtotericin B, amoxicillin, ampicillin, atovaquone, azithromycin, azithromycin, bacitracin, cefazolin, cefepime, cefotaxime, cefotetan, cefpodoxime, ceftazidime, ceftizoxime, ceftriaxone, cefuroxime, cephalexin, chloramphenicol, clotimazole, ciprofloxacin, clarithromycin, clindamycin, dapsone, dicloxacillin, fluconazole, foscarnet, ganciclovir, gatifloxacin, griseofulvin, isoniazid, itraconazole, ketoconazole, metronidazole, nafcillin, neomycin, nitrofurantoin, nystatin, pentamidine, rifampin, rifamycin, valacyclovir, vancomycin, etc. The indications, effective doses, formulations, contraindications, vendors, etc. of these antibiotics are known to one skilled in the art.

Macrolide antibiotics can be administered in a concentration ranging from about 20 μg/ml to about 200 μg/ml (about 0.002%^{w/v} to about 0.02%^{w/v}). Formulations and doses of macrolide antibiotics are described in co-pending U.S. patent application Ser. Nos. 10/667,161 and 10/752,124, each of which is expressly incorporated by reference herein in its entirety.

In addition to a macrolide antibiotic the formulation can also include mycophenolic acid. Such a formulation when prepared as a pharmaceutically acceptable topically administered solution may include about 0.5%^{w/v} to about 10%^{w/v} mycophenolic acid. Preferably, a concentration of macrolide antibiotic and/or mycophenolic acid in a pharmaceutically acceptable topically administered solution may range from about 3%^{w/v} to about 5%^{w/v}. In another embodiment, a concentration of macrolide antibiotic and/or mycophenolic acid in a pharmaceutically acceptable topically administered solution may range from about 1%^{w/v} to about 3%^{w/v}. In another embodiment, a concentration of macrolide antibiotic and/or mycophenolic acid in a pharmaceutically acceptable topically administered solution may range from about 3%^{w/v} to about 10%^{w/v}. In another embodiment, a concentration of macrolide antibiotic and/or mycophenolic acid may range from about 0.1% to about 10% in a topical ocular formulation for treating diabetic retinopathy, age related macular degeneration, or retinitis pigmentosa. In another embodiment, concentrations of macrolide antibiotics and/or mycophenolic acid up to about 2%, up to about 5%, up to about 10%, or exceeding 10% are formulated for topical administration when the compound(s) is bound to a matrix or polymer which slowly releases the compound(s) over time while not exceeding an intraocular concentration of 40 μg/ml.

In a highly preferred form the following formulation may be used: a 1:1 combination of about 0.03%^{w/v} flurbiprofen and a macrolide antibiotic in a concentration and dose sufficient to reduce ocular neovascularization.

In a fifth form, the formulation comprises an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and an inhibitor of a metalloproteinase in a concentration and dose to reduce ocular neovascularization.

Inhibitors of metalloproteinases include naturally occurring proteins such as TIMP-1 that specifically inhibit matrix metalloproteinases, and synthetic metalloproteinase inhibitors such as Batimastat (BB-94) and marimastat (BB-2516) which potently and specifically inhibit metalloproteinase production. These inhibitors degrade the extracellular matrix, promoting tumor invasion and metastasis, but also regulate host defense mechanisms and normal cell function. Selective inhibition is expected to inhibit reactions leading to neovascularization in the inventive formulations and methods. Such metalloproteinase inhibitors are also included in the invention. Among the twenty-four MMPs described, eight have been identified in the cornea, i.e., collagenase I and III (MMP-1 and MMP-13), gelatinase A and B (MMP-2 and -9), stromelysin (MMP-3), matrilysin (MMP-7) and membrane type MMP (MMP-14).

In a highly preferred form the following formulation may be used: a 1:1 combination of about 0.03%^{w/v} flurbiprofen and a metalloproteinase inhibitor in a concentration and dose sufficient to reduce ocular neovascularization.

In an alternate embodiment of the invention the formulation comprises: (a) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization; and (b) a plurality of compounds in a concentration and dose to reduce ocular neovascularization, wherein the compounds are selected from the group consisting of: a tetracycline or a derivative thereof (including CMTs which inhibit MMP activity), a steroid, an antimicrobial, heparin, and/or a metalloproteinase inhibitor.

Where there are a plurality of tetracycline compounds or derivatives thereof (including CMTs which inhibit MMP activity), steroids, antimicrobials, heparin, and/or a metalloproteinase inhibitor compounds employed in the formulation, the preferred compounds and their doses will be those which are described above. In an illustrative form of the invention such a formulation can comprise:
(1) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml;
(2) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a heparin or low molecular weight heparin in a concentration from about 0.01 pg/ml to about 100 mg/ml;
(3) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a macrolide antibiotic such as ascomycin at a concentration from about 20 μg/ml to about 200 μg/ml;
(4) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a heparin or low molecular weight heparin in a concentration from about 0.01 pg/ml to about 100 mg/ml and an macrolide antibiotic such as ascomycin at a concentration from about 20 μg/ml to about 200 μg/ml;
(5) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a heparin or low molecular weight heparin in a concentration from about 0.01 pg/ml to about 100 mg/ml and an inhibitor of a metalloproteinase in a concentration and dose to reduce ocular neovascularization;
(6) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an inhibitor of a metalloproteinase in a concentration and dose to reduce ocular neovascularization;
(7) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and a heparin or low molecular weight heparin in a concentration from about 0.01 pg/ml to about 100 mg/ml; or
(8) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and a macrolide antibiotic such as ascomycin at a concentration from about 20 μg/ml to about 200 μg/ml; or
(9) an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and a heparin or low molecular weight heparin in a concentration from about 0.01 pg/ml to about 100 mg/ml and a macrolide antibiotic such as ascomycin at a concentration from about 20 μg/ml to about 200 μg/ml.

The skilled reader will appreciate that the duration over which any of the formulations of the invention will dwell in the ocular environment will depend, inter alia, on such factors as the pharmacological properties of the compounds employed in the formulation, the concentration of the compound employed, the bioavailability of the compound, the disease to be treated the mode of administration and the preferred longevity of the treatment. Where that balance is struck will often depend on the longevity of the effect required in the eye and the ailment being treated. Formulations prepared according to the invention will preferably have dwell times from hours to many months and possibly years, although the latter time period requires special delivery systems to attain such a duration. Some illustrative forms of such delivery systems are disclosed below. Most preferably the formulations described herein will have a dwell time (ie duration in the eye) of hours (i.e. 1 to 24 hours), days (i.e. 1, 2, 3, 4, 5, 6 or 7 days) or weeks (i.e. 1, 2, 3, 4 weeks). Alternatively, the formulation will have a dwell time of at least a few months such as, 1 month, 2 months, 3 months, with dwell times of greater than 4, 5, 6, 7 to 12 months being achievable.

The precise formulation used in the pharmaceutical formulation of the present invention will vary according to a wide range of commercial and scientific criteria. That is the skilled reader will appreciate that the above formulation of the invention described above may contain other agents. For example the formulations of the invention are preferably prepared using a physiological saline solution as a vehicle. The pH of the formulation may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulation may additionally include at least a pharmaceutically acceptable additive (such as a diluent, carrier, adjunct, excipient or non-toxic, non-therapeutic, non-immunogenic stabilizers and the like). Preferably, the pharmaceutically acceptable additive should be ophthalmologically acceptable, preferably being compatible with the vitreous, and should not leave any vision impairing residue in the eye. Desirably, any pharmaceutically acceptable additive used in the formulation may preferably be suited to the delivery of said pharmaceutical formulation as an intravitreal depot injection.

Any diluent used in the preparation of the pharmaceutically acceptable formulation may preferably be selected so as not to unduly affect the biological activity of the formulation. Examples of such diluents which are especially useful for injectable formulations are water, the various saline, organic or inorganic salt solutions, Ringer's solution, dextrose solution, and Hank's solution.

In addition, the pharmaceutical formulation may include additives such as other buffers, diluents, carriers, adjuvants or excipients. Any pharmacologically acceptable buffer suitable for application to the eye may be used, e.g., tris or phosphate buffers. Other agents may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. The indications, effective doses, formulations, contraindicatons, vendors etc, of the compounds in the formulations are available or are known to one skilled in the art.

These agents may be present in individual amounts of from about 0.001% to about 5% by weight and preferably about 0.01% to about 2%. Suitable water soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 9 and preferably about 4 to about 8. As such the buffering agent may be as much as about 5% on a weight to weight basis of the total formulation. Electrolytes such as, but not limited to, sodium chloride and potassium chloride may also be included in the formulation.

Accordingly in another embodiment, the invention resides in a method for reducing ocular neovascularization comprising the step of: administering to a patient an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization. In a more preferred form of the invention the formulation used in the above method is a formulation described above to reduce neovascularization in the anterior and/or posterior portions of the eye, or in the cornea, retina, choroid, etc.

Administration of the inventive formulation should at least reduce ocular neovascularization. Vessel regression may occur in addition to, or in place of, prevention of further vessel growth or proliferation. As will be appreciated, the cumulative effects may be important in managing diseases such as diabetes, where control of the complicating factors of the disease is as important as control of the underlying pathology to maintain a patient's quality of life.

The route and form of administration of the antiprostaglandin formulation may be any method known to one skilled in the art. Administration may be by topical, subconjunctival, sub-Tenon, and intraocular routes or via ocular implants.

In one embodiment, the formulation is intraocularly injected, for example, into the vitreous. When administering the formulation by intravitreal injection, the active agents should be concentrated to minimise the volume for injection. For injection, a concentration less than about 20 mg/ml may be injected, and any amount may be effective depending upon the factors previously described. Preferably a dose of less than 7 mg/ml is administered, with doses of less than 6 mg/ml, 5 mg/ml, 4 mg/ml 3 mg/ml, 2 mg/ml and 1 mg/ml being more preferred. Sample concentrations include, but are not limited to, about 5 μg/ml to about 50 μg/ml; about 25 μg/ml to about 100 μg/ml; about 100 μg/ml to about 200 μg/ml; about 200 μg/ml to about 500 μg/ml; about 500 μg/ml to about 750 μg/ml; about 500 μg/ml up to 1 mg/ml; etc.

For example, in preparation for injection, topical alcaine may be applied to the ocular surface, followed by 5% povidone iodine. A cotton-tipped applicator soaked in 4% lidocaine is then applied to the injection site, which is 4.0 mm posterior to the limbus in phakic eyes and 3.5 mm posterior to the limbus in pseudophakic eyes. A 27-gauge needle may be used for injection at the superior pars plana. Indirect ophthalmoscopy can be used to confirm proper intravitreal placement of the suspension.

The syringe used in practicing this invention is suitably one which can accommodate a 21 to 30 gauge needle (eg a 23, 24, 25, 26 or 27 gauge needle) and is preferably of a small volume, for example 1.5 mL, or more preferably 0.5 mL. Although it is possible that the needle and syringe may be of the type where the needle is removable from the syringe, it is preferred that the arrangement is of a unitary syringe/needle construction. This would clearly limit the possibility of disengagement of the needle from the syringe. It is also preferred that the arrangement be tamper evident. The formulations of the present invention may therefore be provided in the form of a single unit dose in a pre-prepared syringe, ready for administration.

A suitable style of syringe is, for example, sold under the name of Uniject™ manufactured by Becton Dickinson and Company. In this style of syringe, the material is expelled through the needle into the eye by pressure applied to the sides of a pliable reservoir supplying the needle, rather than by a plunger. As the name implies, the construction of the reservoir and needle forms a single unit.

Topical application of formulations of the invention may be as an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye. Indeed, it can be advantageous to formulate a formulation of the invention as a gel, to minimize loss of the formulation immediately upon administration, as a result, for example, of lacrimation caused by reflex blinking. Although it is preferred that such a formulation exhibit further increase in viscosity or gel stiffness upon administration, this is not absolutely required if the initial gel is sufficiently resistant to dissipation by lacrimal drainage to provide the effective residence time specified herein.

To prepare a topical formulation for the treatment of ophthalmological disorders, a therapeutically effective amount of the formulation of the invention is placed in an ophthalmological vehicle as is known in the art. The amount of the therapeutic compound to be administered and the concentration of the compound in the topical formulations depend upon the diluent, delivery system or device selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

For topical administration, the concentration of antiprostaglandin administered may depend upon the particular patient, the underlying disease and its severity, the dosing frequency, etc., as known to one skilled in the art. Sample concentrations include, but are not limited to, about 0.5 mg/ml to about 2.5 mg/ml, about 1 mg/ml to about 5 mg/ml, about 5 mg/ml to about 10 mg/ml, about 10 mg/ml to about 15 mg/ml, about 15 mg/ml up to 30 mg/ml, etc.

The formulation may also be administered as a slow release formulation, with a carrier formulation such as nanospheres, nanocapsules, microspheres, microcapsules, liposomes, etc., as an intravenous solution or suspension, or in an intraocular injection, as known to one skilled in the art.

A time-release drug delivery system may be administered intraocularly to result in sustained release of the agent over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered through a needle, or may be implanted by suturing within the eye, for example, within the lens capsule. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, New York 2000), the relevant sections of which are incorporated by reference herein in their entirety. For example, a sustained release intraocular implant may be inserted through the pars plana for implantation in the vitreous cavity. An intraocular injection may be into the vitreous (intravitreal), or under the conjunctiva (subconjunctival), or behind the eye (retrobulbar), or under the Capsule of Tenon (sub-Tenon), and may be in a depot form. Other intraocular routes of administration and injection sites and forms are also contemplated and are within the scope of the invention.

Where the administration of two or more active agents is desired, the active agents may be administered as a mixture, as an admixture, in the same formulation, in separate formulations, in extended release formulations, liposomes, microcapsules, or any of the previously described embodiments. A formulation containing two or more agents may be administered topically, or may be injected into the eye, or one active agent may be administered topically and the other agent(s) may be injected.

The method of the present invention may be performed alone, or in combination with one or more other therapies such as photodynamic therapy, laser surgery, laser photocoagulation, or one or more biological or pharmaceutical treatments.

Laser treatment takes a number of forms, depending on the nature of the ophthalmic disorder. Disorders such as myopia may be treated with laser surgery to reshape the cornea (eg. LASIK® surgery), whilst a widely used treatment for disorders such as AMD is laser therapy which is directed to removal or blockage of blood vessels via photodynamic therapy or laser photocoagulation. Laser therapy may further be used to treat or remove neoplasm such as retinoblastomas or pseudogliomas.

The invention therefore resides in a method for reducing ocular neovascularization comprising the step of: administering to a patient a formulation as described above to a patient following corneal surgery (e.g., LASIK® surgery, photorefractive keratectomy (PRK), or other corneal procedures).

Photocoagulation involves the use of a laser to seal leaking blood vessels, slow the growth of abnormal blood vessels and/or destroy new blood vessels within the eye. In addition, the laser can be used to seal the retina to the eye, helping to prevent retinal detachment. For example, focal laser treatment may be applied to microaneurysms identified in diabetic retinopathy.

Photodynamic therapy involves the use of a photoactive drug (eg Visudyne®) and a laser to destroy abnormal blood vessels. Visudyne® is injected into the blood and activated with a laser, effectively destroying the blood vessels. This treatment may require several sessions to be effective. A wide range of theories have been proposed to explain the beneficial effects of retinal laser photocoagulation in delaying retinal angiogenesis, however, the underlying molecular mechanism remains to be elucidated.

The therapeutic effects of laser photocoagulation are thought to be due to the destruction of photoreceptors, the highest oxygen consumers in the retina. Subsequently, these photoreceptors are replaced by glial cells allowing increased oxygen diffusion from the choroid to the inner retina thereby relieving inner retinal hypoxia. This improved oxygenation triggers a two-pronged cascade of events where: (1) constriction of the retinal arteries results in decreased hydrostatic pressure in capillaries and the constriction of capillaries and venules; and (2) the cellular production of VEGF is inhibited. Together, these effects are believed to ultimately result in the inhibition of neovascularization and a decrease in oedema. Cell proliferation and regulation of cellular proteins are induced by the laser photocoagulation, and their therapeutic effect might be an essential part of the physiological response.

However, laser treatment is not always a permanent cure as the blood vessels may begin to grow again, and microaneurysms may reform. Furthermore, laser treatment of abnormal blood vessels cannot be performed on vessels located in certain regions of the retina, such as the central region of the macula.

Therefore, in an embodiment of the invention, where laser treatment of the retina is indicted, administration of an antiprostaglandin such as a flurbiprofen may be carried out by injection before or after the laser treatment. Administration of the anti-oedematous steroid may be before or after laser therapy and may aid in reducing neovascularization prior to laser treatment, or preventing further neovascularization after laser treatment.

Preferably the formulation administered to the patient undergoing laser treatment of any kind is: an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and a tetracycline or a derivative thereof including CMTs which inhibit MMP activity; or an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization and heparin. Alternatively the antiprostaglandin agent may be administered with a tetracycline or a derivative thereof including CMTs which inhibit MMP activity, and a heparin or low molecular weight heparin in a pharmaceutically acceptable form suitable for delivery to the eye to reduce ocular neovascularization.

In a yet another embodiment of the method of the invention, one or more of the formulations described above is administered to a patient in a cyclic tumor treatment regimen to reduce blood vessel growth and proliferation at a tumor site. In this form of the invention, the agents are systemically administered along with standard tumor therapies, so that the agents are rotated, thereby inhibiting blood vessel proliferation throughout the treatment cycle.

In this embodiment, the initial therapy (stage 1) is selected among those presently available: either chemotherapy (e.g., gene therapy, antineoplastic drugs, etc.) or one or more of the following non-chemotherapeutic treatments: radiation therapy (e.g, x-rays, gamma rays, (3 rays, etc.); phototherapy (e.g., photodynamic therapy, photosensitizers); or thermal therapy (e.g., thermal coagulation, hyperthermia, cryotherapy).

Immediately following this initial treatment event, therapy using the inventive formulations is initiated in a rotational cycle. That is, one or more of the formulations described above is administered over the course of one cycle, but the active agents are administered at different stages in the cycle. Each of the agents is administered systemically (e.g., intravenously, orally, etc.) at their highest non-toxic concentration, as known to one skilled in the art. For example, steroids are administered at doses ranging from about 100 mg/ml to about 200 mg/ml. The use of a cyclic rotational administration of each of these vessel-inhibiting agents causes vessel damage at different times and through different processes, thereby maximizing the overall damage to the vessels and inhibiting blood supply to the tumor while conventional tumor therapy occurs (e.g., chemotherapy, radiation therapy, etc.).

The inventive cyclic therapy is initiated by systemic administration of a steroid (stage 1), followed by systemic administration of a formulation containing the same or another steroid and an antiprostaglandin in a concentration from about 0.001%^{w/v} to about 0.5%^{w/v} (stage 2). For example intravenous administration of methylprednisolone (Solu-Medrol®) can be followed by oral administration of prednisone and flurbiprofen. Stage 2 lasts from about one to about two weeks.

Stage 3 follows stage 2, during which a formulation containing an antiprostaglandin in a concentration from about 0.001%^{w/v} to about 0.5%^{w/v} and heparin is administered. Chemotherapeutic drugs may also be administered in stage 3. Stage 3 lasts from about one to about two weeks.

Stage 4 follows stage 3, during which a formulation containing an antiprostaglandin in a concentration from about 0.001%^{w/v} to about 0.5% ^{w/v}, heparin, and macrolide antibiotics are administered. Stage 4 lasts from about one to about two weeks and completes the first treatment cycle, which lasts from about one to about two months.

If additional therapy is required (determined by tumor size, the presence of absence of tumor markers, etc.), further cycle(s) of treatment are initiated. These further cycles may start either with stage 1 and proceed through stages 2, 3, and 4, or may start with stage 2 directly from stage 4 and bypass stage 1. It will be appreciated that any of the agents described herein may be used in any of stages 2, 3, or 4.

Alternatively, any of stages 1 to 4 may also comprise an anti-proliferative (anti-neoplastic agent) such as 5-fluorouracil, methotrexate, vincristine, etoposide, cyclophosphamide, doxorubicin, epirubicin, cytosine arabinoside, actinomycin-D etc. In a further embodiment, anti-proliferative agent(s) may be administered in extra stages between the four stages described above, or may replace one or more of the stages described above. For example, administration of anti-proliferative agents may be performed between stages 2 and 3, or may replace stage 4.

Alternatively, factors such as anti-glial factors and anti-nerve growth factors may be administered with antiprostaglandin for the treatment of ocular neovascularization, either at the same time (eg in a single formulation or two separate formulations) or in alternating cycles of therapy. Such factors may be administered ocularly by topical administration, intravitreal injection etc, or may be administered systemically eg by the parenteral route or orally.

In addition to the above other substances, formulations of the invention may be injected with anti-angiogenic agents designed to block the actions of VEGF on endothelial cells that can be employed in the method of the invention are: (a) Lucentis® developed by Genentech; and (b) Macugen® developed by Eyetech Pharmaceuticals. Lucentis® and Macugen® are compounds that are injected into the vitreous and are potent anti-angiogenic compounds. In a highly preferred form, the pharmaceutical formulation of the invention will comprise a formulation of the invention as described and an anti-angiogenic agent such as Lucentis® or Macugen®.

Lucentis® (ranibizumab), formerly known as rhuFab V2 or AMD-Fab is a humanized, therapeutic anti-VEGF (vascular endothelial growth factor) antibody fragment developed at Genentech to bind and inhibit VEGF, a protein that plays a critical role in angiogenesis (the formation of new blood vessels). Lucentis® is designed to block new blood vessel growth and reduce leakage, which are thought to lead to wet AMD disease progression. When administered in conjunction with pharmaceutical formulations prepared according to the present invention Lucentis® should be administered in either about 300 or about 500 microgram doses for four doses.

Macugen® (pegaptanib sodium, anti-VEGF aptamer or EYE001) developed by Eyetech Pharmaceuticals, consists of a synthetic fragment of genetic material that specifically binds to the VEGF molecule and blocks it from stimulating the receptor on the surface of endothelial cells. When administered in conjunction with pharmaceutical formulations prepared according to the present invention Macugen® should be administered in a dose ranging from either about 0.3 mg to about 3.0 mg every four or six weeks.

In another aspect of the invention, pharmaceutical formulations prepared according to the present invention may be prepared in combination with a glucocorticoid (e.g. prednisolone, prednisone), an oestrogen (e.g. oestrodiol), an androgen (e.g. testosterone) retinoic acid derivatives (e.g. 9-cis-retinoic acid, 13-trans-retinoic acid, all-trans retinoic acid), a vitamin D derivative (e.g. calcipotriol, calcipotriene), a non-steroidal anti-inflammatory agent, a vitamin D derivative, an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein. In still other embodiments of the invention, a mixture of these agents may be used.

Ocular anti-infective agents as described herein include, but are not limited to, penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (amikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscarnet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan), anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), immunosuppressant agents such as cyclosporin A and SK506, and anti-inflammatory or suppressive factors (inhibitors), and inhibitors of proteolytic enzymes such as plasminogen activator inhibitors.

Doses for topical and sub-conjunctival administration of the above agents, as well as intravitreal dose and vitreous half-life may be found in Intravitreal Surgery Principles and Practice, Peyman G A and Shulman, J Eds., 2nd edition, 1994, Appleton-Longe, the relevant sections of which are expressly incorporated by reference herein.

The present invention also provides the use of an antiprostaglandin in a concentration from about 0.001%^{w/v} to about 0.5% ^{w/v}, characterized in that said compound is at a substantially neutral pH in a pharmaceutically acceptable form suitable for delivery to the eye to reduce ocular neovascularization in the manufacture of a medicament for the treatment of ocular neovascularization.

The invention further provides the use of a formulation as herein described in the preparation of a medicament for the treatment of ocular neovascularization.

The invention also provides the use of a formulation as herein described as well as anti-angiogenic agents designed to block the actions of VEGF on endothelial cells in the preparation of a medicament for the treatment of ocular neovascularization.

### EXAMPLES

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

### Example 1

Artificial corneal burns were induced in thirty-two eyes belonging to thirty-two Long Evans rats to determine the effects of doxycycline or another tetracycline derivative and low molecular weight heparin, doxycycline or another tetracycline derivative, and flurbiprofen, or flurbiprofen and low molecular weight heparin, on corneal neovascularization. All the eyes were examined to exclude any eyes with corneal scars and/or neovascularization prior to induction. More specifically, topical administration of the described two drug combination was administered twice a day to rats in which corneal burns had been artificially induced by application of silver nitrate (70%) and potassium nitrate (30%).

Neovascularization was induced in all eyes using silver nitrate cauterization. The animals were first anesthetized by intraperitoneal injection of a mixture of ketamine hydrochloride (25 mg/kg) with xylazine hydrochloride (5 mg/kg). The cornea was then anesthetized by a drop of 0.5% proparacaine and allowed to dry. One cornea of each animal was cauterized by pressing an applicator stick (diameter of 1.8 mm) coated with 75% silver nitrate/25% potassium nitrate (Arzol Chemical Co., Keen, N.H.) to the central cornea for ten seconds (using a stopwatch) under the operating microscope. Excess silver nitrate was removed by rinsing the eyes with balanced salt solution. To increase the reproducibility of the injuries, one investigator cauterized all animals.

Following cauterization, the animals were randomly divided into four groups to eliminate any potential bias in the degree of injury with the different groups. Group 1 (number of animals, n=8) received a 1:1 combination of 0.03% flurbiprofen sodium ophthalmic solution (Allergan, Irvine Calif.) and 10 mg/ml low molecular weight heparin (Enoxaparin, Aventis Pharmaceuticals Inc., Bridgewater N.J.); an actual concentration of 0.015% flurbiprofen with 5 mg/ml low molecular weight heparin. Group 2 (n=8) received a 1:1 combination of 0.03% flurbiprofen sodium ophthalmic solution and 20 mg/ml doxycycline (American Pharmaceutical Partners, Schaumburg Ill.); an actual concentration of 0.015% flurbiprofen with 10 mg/ml doxycycline. Group 3 (n=8) received a 1:1 combination of 20 mg/ml doxycycline and 10 mg/ml low molecular weight heparin; an actual concentration of 10 mg/ml doxycycline with 5 mg/ml low molecular weight heparin. Group 4 (n=8) received balanced salt solution (control). The drops were applied topically immediately after cauterization; treatments were administered two times per day for seven days.

The presence of new vessels (neovascularization) and the extent of new vessel formation was assessed by slit lamp photography and histology. Inhibition of vessel proliferation was evaluated by measuring vessel progression from the outer cornea (corneal limbus) into the cornea. As previously described, it will be appreciated that any reduction of new vessel proliferation and/or regression of existing vessels is therapeutic, and that complete inhibition and/or regression is not required, and also that reduction includes regression of existing vessels.

The extent of corneal neovascularization was determined by slit lamp microscopy with photography (SL-7E, Topcon, Tokyo Japan) on day seven after cauterization. The animals were euthanized in a carbon dioxide chamber under deep general anesthesia. The eyes were enucleated and fixed in 10% formaldehyde. After fixation for 24 hours, the eyes were removed from the fixative and corneas were dehydrated and sectioned. The corneas were then soaked in xylene and paraffin, later they were embedded in paraffin and cut at 1 pm for staining with hematoxylin-eosin (H&E) for light microscopy.

Corneal neovascularization was assessed by scanning (Cano scan 9900F, Canon, Tokyo Japan) the slit lamp photographs into high resolution digital images. The percentage area of corneal neovascularization was determined by outlining the areas with corneal vessels and comparing these to the total corneal surface using image j software (Wayne Rasband at the Research Services Branch, National Institute of Mental Health, Bethesda Md.). The percentage area of the cornea covered by the corneal scar in each eye was also determined. A drawing of corneal blood vessels was made to compare with digital photos and ensure that no vascular area was omitted during calculation of percent area.

Statistical analysis was performed using Statistical Analysis System (SPSS 11.5) software. The difference between the groups was determined using Mann-Whitney U Analysis test; a p value less than 0.05 was considered significant.

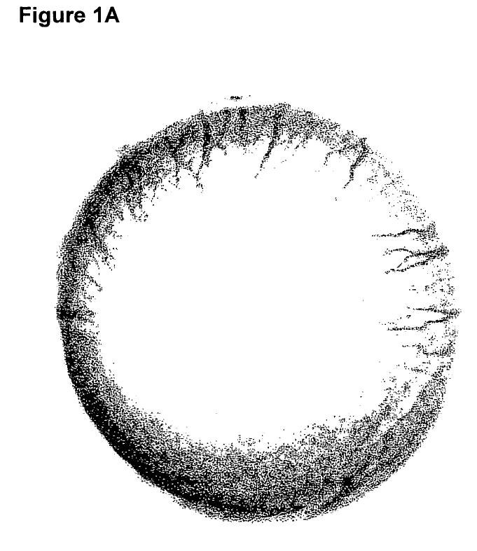

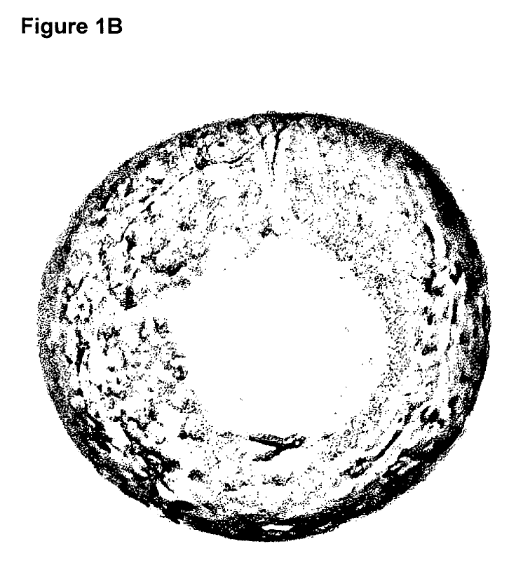

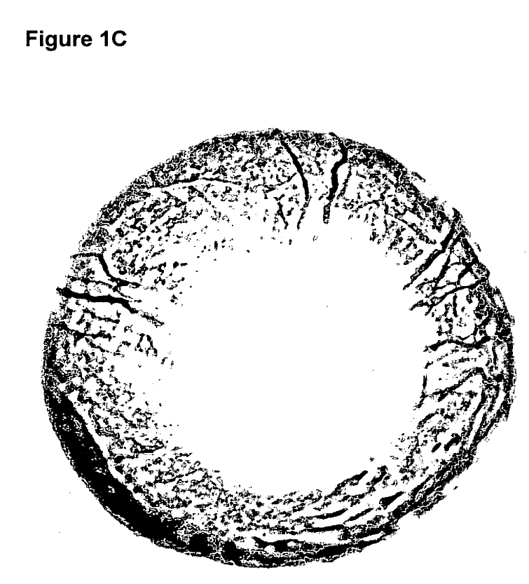

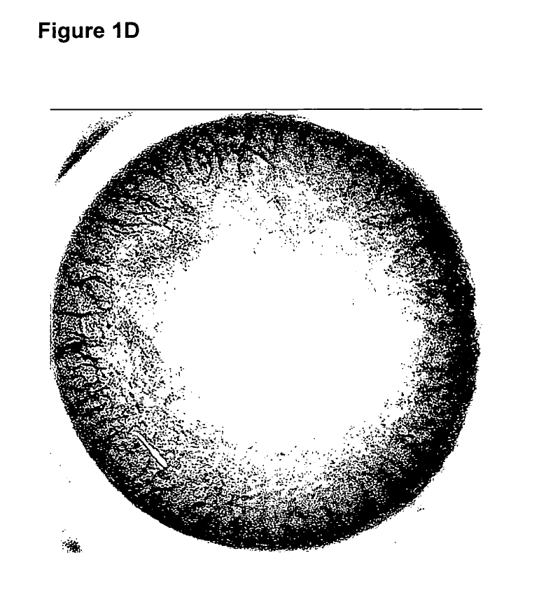

Representative digitally enhanced slit lamp photographs of the cornea seven days after induction of corneal burn in treated eyes are shown in FIGS. 1A-1 D. After administration of flurbiprofen and low molecular weight heparin, neovascularization was prominent but was less than in the control group (FIG. 1A). After administration of flurbiprofen and doxycycline, there was minimal neovascularization (FIG. 1B). After administration of doxycycline and low molecular weight heparin there was moderate neovascularization (FIG. 1C). After administration of a balanced salt solution (control), there was extensive neovascularization (FIG. 1D).

The percentage of corneal neovascularization, corneal scar size and burn intensity was determined for all eyes using J image on the digitized slit lamp photographs.

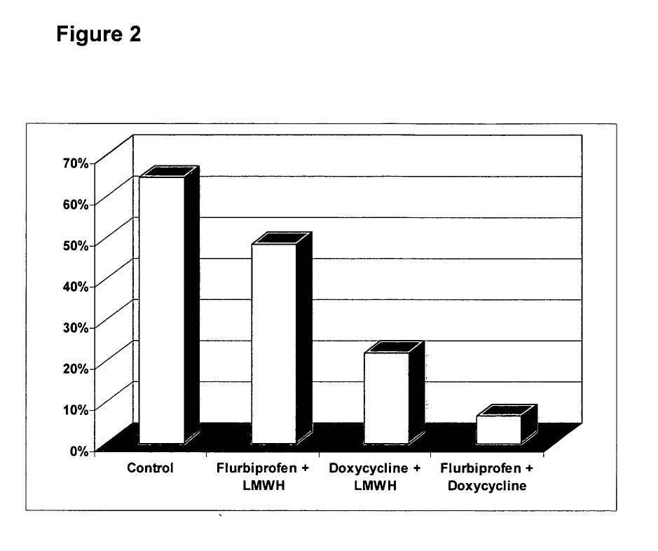

There was no statistically significant difference in the corneal scar size and burn intensity in any of the eyes. The mean percentage neovascularization for eyes administered flurbiprofen and low molecular weight heparin was 48.5±13.1. The mean percentage neovascularization for eyes administered flurbiprofen and doxycycline was 6.6±5.5. The mean percentage neovascularization for eyes administered doxycycline and low molecular weight heparin was 22.0±27.6. The mean percentage neovascularization for the control group was 64.6±9.9. Data are summarized in FIG. 2.

Neovascularization in each treatment group was statistically compared with the control and among the treatment groups using the Mann Whitney U analysis. Administration of flurbiprofen and doxycycline, and low molecular weight heparin and doxycycline, showed significantly lower corneal neovascularization when compared to the control group (p<0.05). Although administration of flurbiprofen and low molecular weight heparin showed a trend towards inhibition of corneal neovascularization when compared to the control, the inhibition was not significant (p=0.105).

When the groups were compared to each other, there was no significant difference between administration of low molecular weight heparin and doxycycline, nor was there a significant difference between administration of flurbiprofen and doxycycline (p=0.355). Similarly there was no significant difference between administration of low molecular weight heparin and doxycycline, and administration of flurbiprofen and low molecular weight heparin (p=0.069). There was, however, a significant difference between administration of flurbiprofen and doxycycline, and administration of flurbiprofen and low molecular weight heparin (p=0.02).

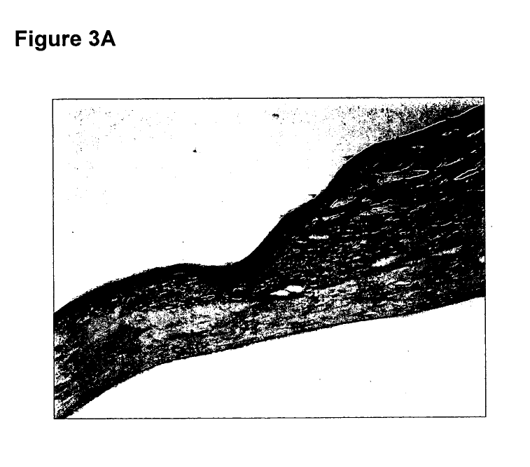

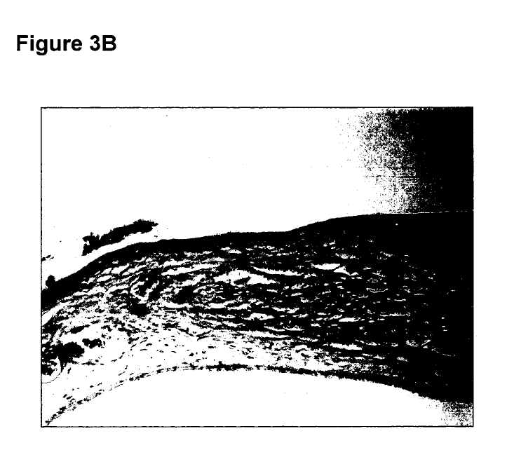

Histological preparations of eyes from each of the treatment groups were stained with hematoxylin and eosin and examined using light microscopy. The results are shown in FIG. 3. FIG. 3A is an eye administered flurbiprofen and doxycycline; there were no vessels in the central stroma. FIG. 3B is an eye administered normal saline; extensive neovascularization involved the central corneal stroma.

Light microscopy evaluation of the histological preparations from the different groups was consistent with the slit lamp evaluation. Although all the treatment groups had more of an anti-angiogenic action when compared to the control, the group to which flurbiprofen and doxycycline was administered had the greatest effect. This indicated that flurbiprofen and doxycycline provided the greatest inhibition of neovascularization among the groups evaluated.

Each of the three possible two drug combinations of flurbiprofen, doxycycline, and low molecular weight heparin were effective in inhibiting corneal neovascularization when compared to control. The combinations of doxycycline and low molecular weight heparin, and doxycycline and flurbiprofen, were more effective than the combination of flurbiprofen and low molecular weight heparin. Flurbiprofen is a non-steroidal anti-inflammatory agent that inhibits the synthesis of prostaglandins. Prostaglandins are produced in corneal wound healing and angiogenesis. Thus, flurbiprofen suppresses actively proliferating corneal vessels.

Flurbiprofen (0.03%^{w/v}) and low molecular weight heparin (10 mg/ml), administered as individual agents, did not significantly decrease corneal neovascularization in this model (data not shown). Doxycycline did significantly inhibit (p<0.05%) corneal neovascularization when administered at 20 mg/ml and not when administered at 10 mg/ml.

As previously described, combinations of flurbiprofen, low molecular weight heparin and doxycycline were more effective than when these agents are used individually at similar or higher concentrations. Without being bound by a particular theory, a mechanism may be that each agent has a different mode/site of action in the angiogenesis process. The combination may decrease the individual side-effects of the agents and target angiogenesis at different steps. This may decrease the neovascularization response and avoid use of higher concentrations of potentially therapeutic agents with ocular side effects.

### Example 2

Forty eyes belonging to forty male Long Evans pigmented rats weighing 200 to 250 g were divided into different groups for this study. All of the procedures involving animals were conducted in accordance with the Association for Research in Vision and Ophthalmology resolution on the use of animals in research. The studies were approved by the Institutional Animal Care and Use Committee of Tulane University Health Sciences Center.

Prior to all procedures, the rats were anesthetized by using intraperitoneal injection of ketamine hydrochloride (25 mg/kg) with xylazine hydrochloride (5 mg/kg). After using proparacaine hydrochloride as a topical anaesthetic agent one cornea of each animal was cauterized by pressing an applicator stick (with a diameter of 1.8 mm) coated with 75% silver nitrate/25% potassium nitrate (Arzol Chemical Co., Keen, N.H.) to the central cornea for 10 seconds. To increase the reproducibility of the injuries, one investigator cauterized all animals. Excess silver nitrate was removed by rinsing the eyes with 5 ml of balanced salt solution and then gently blotting the eyes with tissue paper.

In the first group (n=7) topical normal saline was used to ensure that chemical burns were of sufficient depth and degree to result the desired neovascular response and to compare the results of other groups with them. Group two (n=6) was treated with topical ascomycin (A. G. Scientific, Inc., San Diego, Calif.) solution made by dilution to the concentration of 50 μg/ml. In group three (n=6) flurbiprofen sodium ophthalmic solution (0.03%) (Allergan, Irvine, Calif.) was used. In group four (n=7) doxycycline solution with the concentration of 20 mg/ml made by dilution of doxycycline vials (American Pharmaceutical Partners, Schaumburg, Ill.) was instilled topically. Group five (n=7) was also treated with topical instillation of low molecular weight heparin solution (Enoxaparin sodium injection, Aventis Pharmaceuticals Inc., Bridgewater, N.J.) diluted to 10 mg/ml. The last group (n=7) received topical instillation of triamcinolone acetonide (4 mg/ml) (Bristol-Myers Squibb Company, Princeton, N.J.).

Those treatments were applied immediately after cauterization in eyes in each group. Treatment (topical) was continued two times daily at equal intervals for 7 days. An evaluation of corneal burn intensity such as described by Mahoney was made by observing the amount of elevation above corneal surface and if there was no elevation the animal was excluded. Extent of the scar was also evaluated by calculating the percentage of corneal surface occupied by the scar.

Drops were applied a few seconds apart allowing the animals to blink between drops. Corneal photographs were taken at the slit-lamp microscope (SL-7E, Topcon, Tokyo Japan) under general anaesthesia on the 7^{th} day. Inhaled carbon dioxide was then used to sacrifice the rats while under deep anaesthesia. Cauterized eyes were enucleated and fixed in formaldehyde 10% for one week. Corneal sections were prepared from all the eyes and histological exam using H&E staining was performed.

The colour slides of the cornea were converted to digital images using a scanner (Cano scan 9900F, Canon, Tokyo, Japan). The area of each cornea and its neovascularization was measured separately by using image j software (Wayne Rasband at the Research Services Branch, National Institute of Mental Health, Bethesda, Md.) and percentage of cornea occupied by vessels and corneal scar was calculated separately. A drawing of corneal blood vessels was made by one of investigators to compare with digital photos and to be sure that no vascular area is missing during calculation of percent area. Statistical analyses of neovascular and scar percent area in each group were performed using a General Linear Models (GLM) procedure with a Tukey's studentized range test which controls the Type I experimentwise error rate (SAS version 8,02 Cary, N.C.). Statistical significance was set at p≦0.05.

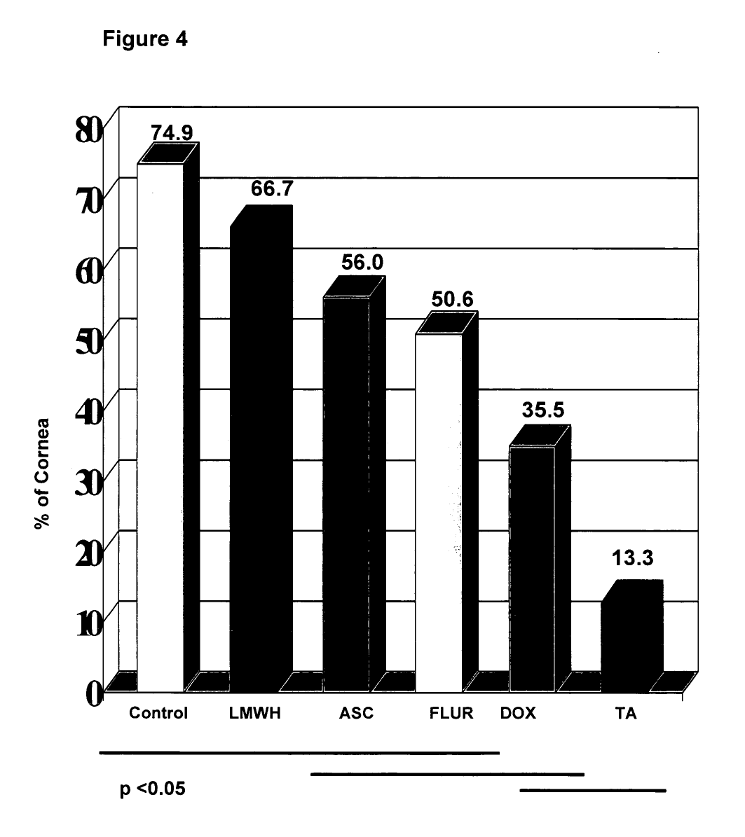

The percentage of burn scar area and neovascularization (relative to total corneal area) in each animal is shown in Table 1. The mean of percent area in the control group was 74.9%±9.2%, while it was 66.7%±9.9%, 56.0%±22.4%, 50.5%±18.7%, 35.5%±29.1%, and 13.3%±7.1% respectively in the LMWH, ascomycin, flurbiprofen, doxycycline, and triamcinolone groups (FIG. 4).

TABLE 1
Percent area of neovascularization and percent area of scar
in each cornea of different animal groups.

| | Percent Area Of | Percent Area |
|---|---|---|
| | Neovascularization | Of Scar |
| Agent Used | Mean +/− SD | Mean +/− SD |
| Normal saline | 74.9 +/− 9.2 | 17.3 +/− 3.5 |
| LMWH | 66.7 +/− 9.9 | 15.5 +/− 2.2 |
| Ascomycin | 56.0 +/− 22.4 | 16.7 +/− 8.2 |
| Flurbiprofen | 50.6 +/− 18.7 | 18.8 +/− 5.2 |
| Doxycycline | 35.5 +/− 29.1 | 16.7 +/− 2.8 |
| Triamcinolone | 13.3 +/− 7.2 | 17.3 +/− 2.6 |

There were no statistically significant differences in NV area among the control group and the LMWH, ascomycin and flurbiprofen groups. There were also no significant differences among the ascomycin, flurbiprofen and doxycycline groups or between the doxycycline and triamcinolone groups. There was a significant reduction in NV area in the doxycycline and triamcinolone groups compared to the control group and the LMWH group and the triamcinolone group also demonstrated a significant reduction in NV area compared to ascomycin and flurbiprofen groups.

There was no significant difference in percentage of burn scar area between the control and any of the study groups.

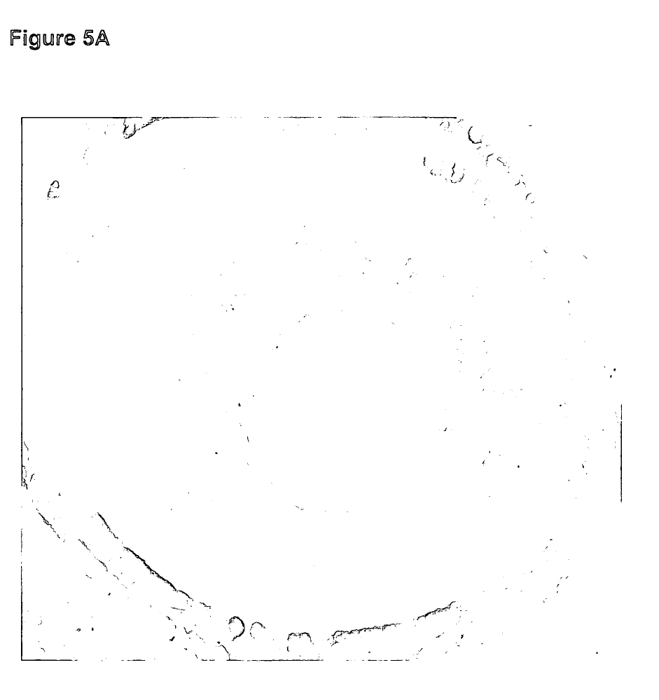

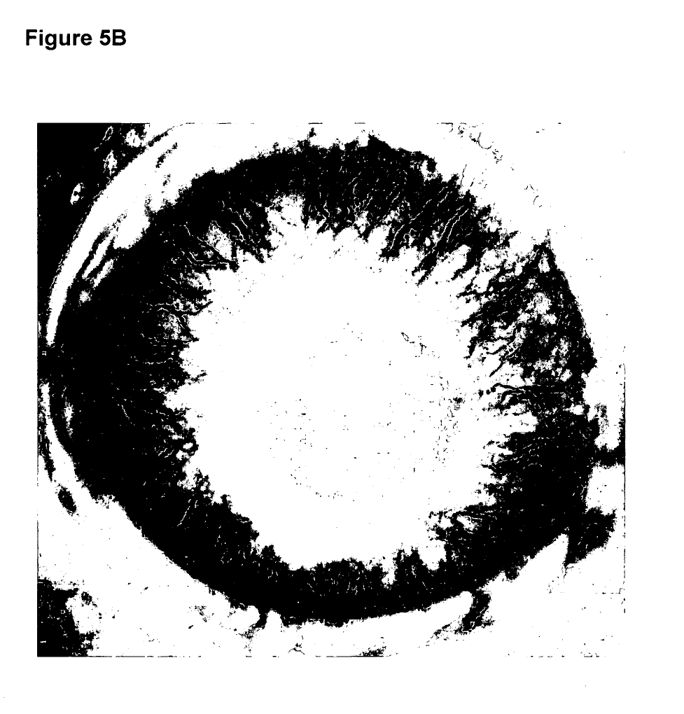

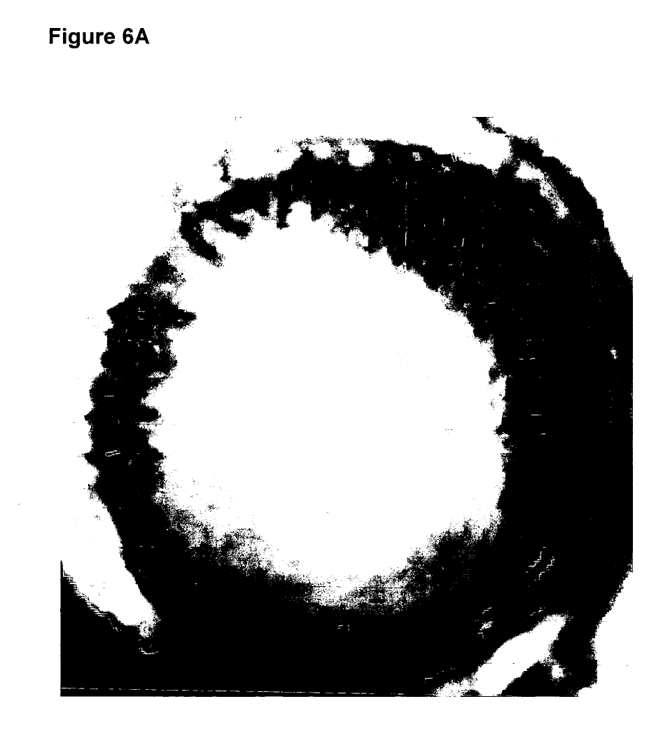

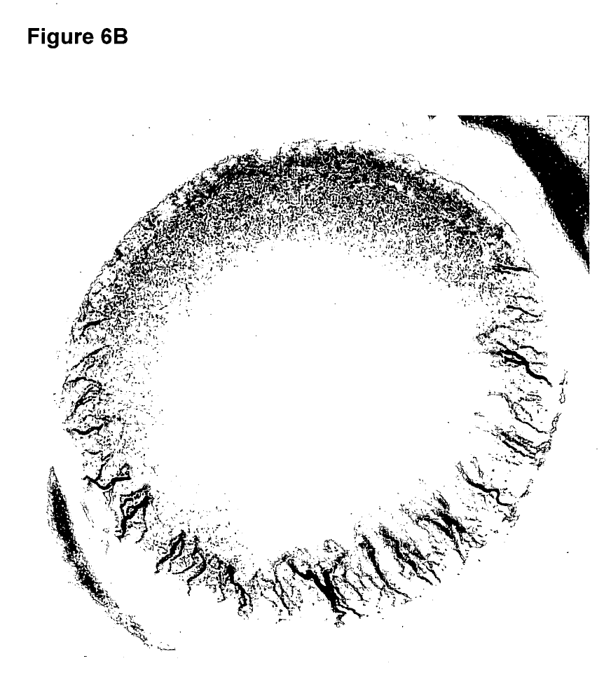

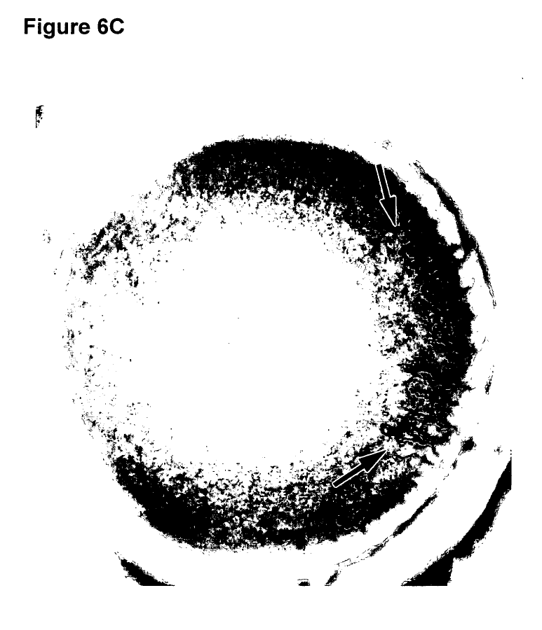

A representative corneal picture in the control group is shown in FIGS. 5A and 5B. FIG. 6A is a digitally enhanced slit lamp photograph of the cornea seven days after induction of corneal burn in eyes treated with flurbiprofen (neovascularization is quite prominent in this group). FIG. 6B is a digitally enhanced slit lamp photograph of the cornea seven days after induction of corneal burn in eyes treated with doxycycline (neovascularization is less prominent than in control group). FIG. 6C is a digitally enhanced slit lamp photograph of the cornea seven days after induction of corneal burn in eyes treated with triamcinolone acetonide (arrows circumscribe the relatively small neovascular area).

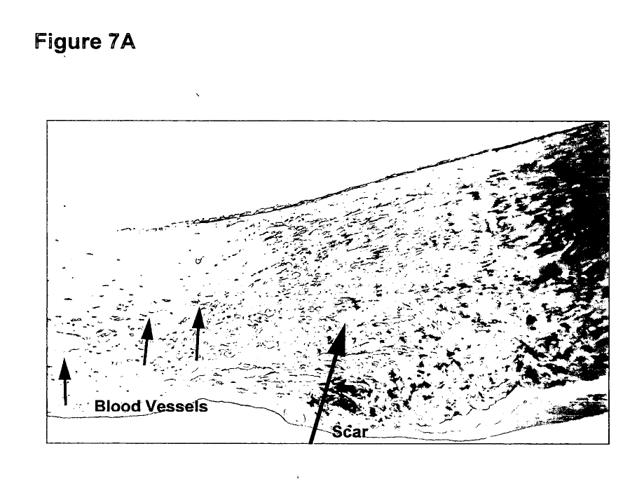

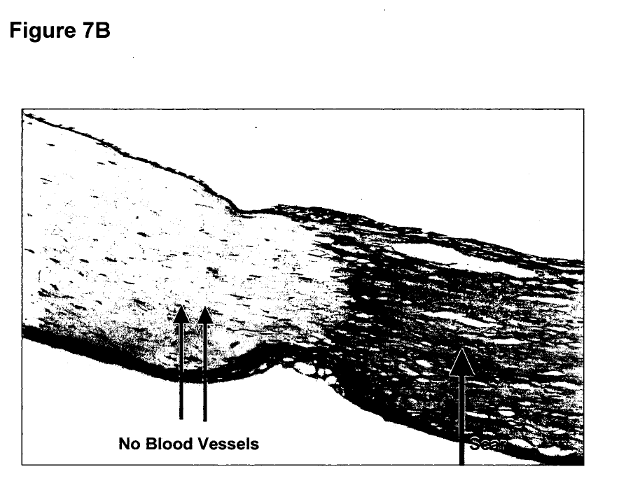

Representative histologic sections of the control and triamcinolone groups are shown in FIG. 7. FIG. 7A is a photograph of a histopathology preparation of the corneal burn in a control eye treated with normal saline, showing corneal scar (large arrow) and new vessels (small arrows) in the corneal stroma. H&E 100X. FIG. 7B is a photograph of a histopathology preparation of the corneal burn in an eye treated with triamcinolone acetonide (double arrows point to avascular stroma). Note extensive neovascularization of the corneal stroma in FIG. 7A compared to FIG. 7B. H&E 100X.

The invention is further described by the following numbered paragraphs:
1. A formulation for the treatment of ocular neovascularization comprising an antiprostaglandin in a concentration and dose suitable for treating ocular neovascularization, characterized in that said compound is in a pharmaceutically acceptable form suitable for delivery to the eye.
2. A formulation for the treatment of ocular neovascularization comprising an antiprostaglandin in a concentration and dose suitable for treating ocular neovascularization, characterized in that said antiprostaglandin is at a substantially neutral pH in a pharmaceutically acceptable form suitable for delivery to the eye.
3. An ocular pharmaceutically acceptable formulation according to paragraph 1 wherein the antiprostaglandin is selected from the group consisting of: flurbiprofen, indomethacin, ketorolac, tromethamine, meclofenamate, flurbiprofen, and compounds in the pyrrolo-pyrrole group of non-steroidal ant-inflammatory drugs.
4. An ocular pharmaceutically acceptable formulation according to paragraph 1 wherein the antiprostaglandin is flurbiprofen.
5. An ocular pharmaceutically acceptable formulation according to paragraph 1 wherein the antiprostaglandin is in a concentration from about 0.001%^{w/v} to about 0.5%^{w/v}, characterized in that said compound is at a substantially neutral pH in a pharmaceutically acceptable form suitable for delivery to the eye to reduce ocular neovascularization.
6. An ocular pharmaceutically acceptable formulation according to paragraph 5 wherein the antiprostaglandin is present in a concentration range of about about 0.01%^{w/v} to about 0.1%^{w/v}.
7. An ocular pharmaceutically acceptable formulation according to paragraph 5 wherein the antiprostaglandin is present in a concentration range of about 0.01%^{w/v} to about 0.05%^{w/v}.
8. An ocular pharmaceutically acceptable formulation according to paragraph 27 wherein the low molecular weight heparin is present in a concentration of 0.03%^{w/v}.
9. A formulation for the treatment of ocular neovascularization according to paragraph 1 wherein formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization.
10. A formulation for the treatment of ocular neovascularization according to paragraph 9 wherein the concentration of the tetracycline or derivative thereof, may range from about 1 pg/ml to about 40 mg/ml.
11. A formulation for the treatment of ocular neovascularization according to paragraph 9 wherein the tetracycline derivative is present at doses up to about 200 μg when the formulation is administered intravitreally.
12. A formulation for the treatment of ocular neovascularization according to paragraph 9 wherein the tetracycline derivative is present at doses in the range of about 1 pg/ml to about 2 mg/ml when administered intraocularly.
13. A formulation for the treatment of ocular neovascularization according to any one of paragraphs 9 to 12 wherein the tetracycline or a derivative thereof is selected from the group consisting of: doxycycline, demeclocycline, minocycline, oxytetracycline, lymecycline, or a chemically modified tetracycline.
14. A formulation for the treatment of ocular neovascularization according to paragraph 13 wherein the chemically modified tetracycline is selected from the group consisting of CMT-315, CMT-3, CMT-8, CMT-308 and 6-demethyl-6-deoxy-4-dedimethylamino tetracylcine (COL-3).
15. A formulation for the treatment of ocular neovascularization according to paragraph 14 wherein the tetracycline derivative is doxycycline.
16. A formulation according to paragraph 15 wherein the concentration of doxycycline is between about 0.01 μg/ml to about 30 mg/ml.
17. A formulation according to paragraph 15 wherein the concentration of doxycycline is between about 0.05 mg/ml to about 10 mg/ml.
18. A formulation according to paragraph 15 wherein the concentration of doxycycline is between about 1 mg/ml to about 20 mg/ml.
19. A formulation for the treatment of ocular neovascularization according to paragraph 1 wherein the formulation also includes a steroid at a concentration from about 0.1 mg/ml to about 40 mg/ml.
20. A formulation according to paragraph 19 wherein the steroid is selected from the group consisting of: triamcinolone, triamcinolone acetate, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate.
21. A formulation according to paragraph 20 wherein the steroid is an 11-substituted-16α,17α-substituted methylenedioxy steroid.
22. A formulation according to paragraph 20 wherein the steroid is 9-fluoro-11,21-ihydroxy-16,17-[1-methylethylidinebis(oxy)]pregna-1,4-diene-3,20-dione.
23. A formulation according to paragraph 19 wherein the steroid concentration is about 0.1 mg/ml to about 40 mg/ml.
24. A formulation according to paragraph 19 wherein the steroid concentration is about 1 mg/ml to about 20 mg/ml.
25. A formulation according to paragraph 19 wherein the steroid concentration is about 20 mg/ml to about 30 mg/ml.
26. A formulation according to paragraph 19 wherein the steroid concentration is about 20 mg/ml to about 40 mg/ml.
27. A formulation for the treatment of ocular neovascularization according to paragraph 1 wherein the formulation also includes heparin in a concentration and dose sufficient to reduce ocular neovascularization.
28. A formulation according to paragraph 27 wherein the heparin is low molecular weight heparin.
29. A formulation according to paragraph 27 or 28 wherein the concentration of heparin is from about 0.01 pg/ml to about 30 mg/ml.
30. A formulation for the treatment of ocular neovascularization according to paragraph 1 wherein the formulation also includes an antimicrobial in a concentration from about 20 μg/ml to about 200 μg/ml.
31. A formulation according to paragraph 30 wherein the antimicrobial is a macrolide antibiotic.
32. A formulation according to paragraph 31 wherein the macrolide antibiotic is selected from the group consisting of: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.
33. A formulation according to paragraph 31 wherein the macrolide antibiotic is ascomycin.
34. A formulation for the treatment of ocular neovascularization according to paragraph 1 wherein the formulation also includes an inhibitor of a metalloproteinase in a concentration and dose to reduce ocular neovascularization.
35. A formulation according to paragraph 36 wherein the inhibitor of a metalloproteinase is selected from the group consisting of: naturally occurring proteins such as TIMP-1 that specifically inhibit matrix metalloproteinases, and synthetic metalloproteinase inhibitors including Batimastat (BB-94) and marimastat (BB-2516).
36. A formulation according to paragraph 37 wherein the inhibitor of a metalloproteinase is selected from the group consisting of: inhibitors of collagenase I, II and III (MMP-1, MMP-8 and MMP-13), inhibitors of gelatinase A and B (MMP-2 and -9), inhibitors of stromelysin (MMP-3), inhibitors of matrilysin (MMP-7) and inhibitors of membrane type MMP (MMP-14).
37. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a steroid such as triamcinolone acetonide at a concentration from about 0.1 mg/ml to about 40 mg/ml and (c) a tetracycline or a derivative thereof (including CMTs which inhibit MMP activity) such as doxycycline at a concentration from about 0.01 pg/ml to about 30 mg/ml.
38. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a steroid such as triamcinolone acetonide at a concentration from about 0.1 mg/ml to about 40 mg/ml and (c) a heparin or low molecular weight heparin in a concentration from about 0.01 pg/ml to about 100 mg/ml.
39. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a heparin or low molecular weight heparin in a concentration from about 0.01 pg/ml to about 100 mg/ml and (c) a macrolide antibiotic such as ascomycin at a concentration from about 20 μg/ml to about 200 μg/ml.
40. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a heparin or low molecular weight heparin in a concentration from about 0.01 pg/ml to about 100 mg/ml and (c) a tetracycline or a derivative thereof (including CMTs which inhibit MMP activity) such as doxycycline at a concentration from about 0.01 pg/ml to about 30 mg/ml.
41. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a heparin or low molecular weight heparin in a concentration from about 0.01 pg/ml to about 100 mg/ml and (c) an inhibitor of a metalloproteinase in a concentration and dose to reduce ocular neovascularization.
42. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a steroid such as triamcinolone acetonide at a concentration from about 0.1 mg/ml to about 40 mg/ml and (c) an inhibitor of a metalloproteinase in a concentration and dose to reduce ocular neovascularization.
43. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a steroid such as triamcinolone acetonide at a concentration from about 0.1 mg/ml to about 40 mg/ml and (c) a macrolide antibiotic such as ascomycin at a concentration from about 20 μg/ml to about 200 μg/ml.
44. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a tetracycline or a derivative thereof (including CMTs which inhibit MMP activity) such as doxycycline at a concentration from about 0.01 pg/ml to about 30 mg/ml and (c) an inhibitor of a metalloproteinase in a concentration and dose to reduce ocular neovascularization.
45. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a macrolide antibiotic such as ascomycin at a concentration from about 20 μg/ml to about 200 μg/ml and (c) an inhibitor of a metalloproteinase in a concentration and dose to reduce ocular neovascularization.
46. An ocular pharmaceutically acceptable formulation comprising: (a) an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization (b) a tetracycline or a derivative thereof (including CMTs which inhibit MMP activity) such as doxycycline at a concentration from about 0.01 pg/ml to about 30 mg/ml (c) a steroid such as triamcinolone acetonide at a concentration from about 0.1 mg/ml to about 40 mg/ml and (d) a heparin or low molecular weight heparin in a concentration from about 0.01 pg/ml to about 100 mg/ml.
47. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a tetracycline or a derivative thereof (including CMTs which inhibit MMP activity) such as doxycycline at a concentration from about 0.01 pg/ml to about 30 mg/ml (c) a steroid such as triamcinolone acetonide at a concentration from about 0.1 mg/ml to about 40 mg/ml and (d) a macrolide antibiotic such as ascomycin at a concentration from about 20 μg/ml to about 200 μg/ml.
48. An ocular pharmaceutically acceptable formulation comprising: (a) an antiprostaglandin in a concentration from about 0.001%^{w/v} to about 0.5% ^{w/v}, characterized in that said antiprostaglandin is at a substantially neutral pH in a pharmaceutically acceptable form suitable for delivery to the eye to reduce ocular neovascularization (b) a tetracycline or a derivative thereof (including CMTs which inhibit MMP activity) such as doxycycline at a concentration from about 0.01 pg/ml to about 30 mg/ml (c) a steroid such as triamcinolone acetonide at a concentration from about 0.1 mg/ml to about 40 mg/ml (d) a heparin or low molecular weight heparin in a concentration from about 0.01 pg/ml to about 100 mg/ml and (e) a macrolide antibiotic such as ascomycin at a concentration from about 20 μg/ml to about 200 μg/ml.
49. A method for treating ocular neovascularization comprising the step of: administering to a patient an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization.
50. A method for treating ocular neovascularization comprising the step of: administering to a patient a formulation as defined in paragraphs 1 to 49 for sufficient time to treat the ocular neovascularization.
51. A method for reducing ocular irritation comprising the step of: administering to a patient a formulation as defined in anyone of paragraph 1 to 49 following corneal surgery.
52. A method for treating ocular neovascularization comprising the step of: administering one or more of the formulations defined in paragraphs 1 to 49 to a patient in a cyclic tumor treatment regimen to reduce blood vessel growth and proliferation at a tumor site.
53. A method according to anyone of paragraphs 50 to 52 which also includes the step of administering an anti-angiogenic agent designed to block the actions of VEGF on endothelial cells.
54. A method according to anyone of paragraphs 53 wherein the anti-angiogenic agent is a rhuFab V2 or a humanized AMD-Fab or an anti-VEGF aptamer.
55. Use of an antiprostaglandin in a concentration from about 0.001%^{w/v} to about 0.5%^{w/v}, characterized in that said compound is at a substantially neutral pH in a pharmaceutically acceptable form suitable for delivery to the eye to reduce ocular neovascularization in the manufacture of a medicament for the treatment of ocular neovascularization.
56. Use of a formulation as defined in any one of paragraphs 1 to 49 in the preparation of a medicament for the treatment of ocular neovascularization.
57. Use of a formulation as defined in anyone of paragraphs 1 to 49 as well as anti-angiogenic agent designed to block the actions of VEGF on endothelial cells in the preparation of a medicament for the treatment of ocular neovascularization.
58. A use according to paragraph 57 wherein the anti-angiogenic agent is a rhuFab V2 or a humanized AMD-Fab or an anti-VEGF aptamer.
59. An ocular pharmaceutically acceptable formulation according to paragraph 1 substantially as herein described.
60. An ocular pharmaceutically acceptable formulation according to paragraph 1 substantially as herein described in the examples.
61. A method according to paragraph 49 substantially as herein described.
62. A method according to paragraph 49 substantially as herein described in the examples.
63. A use according to paragraph 55 substantially as herein described.
64. A use according to paragraph 55 substantially as herein described in the examples.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention, as defined by the following claims.

1. A formulation for the treatment of ocular neovascularization comprising an antiprostaglandin in a concentration and dose suitable for treating ocular neovascularization, characterized in that said antiprostaglandin is in a pharmaceutically acceptable form suitable for delivery to the eye and optionally, said antiprostaglandin is at a substantially neutral pH, wherein optionally
(a) the antiprostaglandin is selected from the group consisting of flurbiprofen, indomethacin, ketorolac, tromethamine, meclofenamate, flurbiprofen, and compounds in the pyrrolo-pyrrole group of non-steroidal anti-inflammatory drugs or
(b) the antiprostaglandin is flurbiprofen or
(c) the antiprostaglandin is in a concentration from about 0.001%^{w/v} to about 0.5%^{w/v}, characterized in that said compound is at a substantially neutral pH in a pharmaceutically acceptable form suitable for delivery to the eye to reduce ocular neovascularization or
(d) the antiprostaglandin is present in a concentration range of about about 0.01%^{w/v} to about 0.1%^{w/v} or
(e) the antiprostaglandin is present in a concentration range of about 0.01%^{w/v} to about 0.05%^{w/v} or
(f) the formulation formulation also includes heparin in a concentration and dose sufficient to reduce ocular neovascularization, and wherein the heparin is low molecular weight heparin or is from about 0.01 pg/ml to about 30 mg/ml or the low molecular weight heparin is present in a concentration of 0.03%^{w/v} or
(g) the formulation also includes an antimicrobial in a concentration from about 20 μg/ml to about 200 μg/ml, wherein the antimicrobial is a macrolide antibiotic or
(h) the macrolide antibiotic of (g) is selected from the group consisting of tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide or
(i) the macrolide antibiotic of (g) is ascomycin.

2. A formulation for the treatment of ocular neovascularization according to claim 1 wherein formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and optionally wherein
(a) the concentration of the tetracycline or derivative thereof, may range from about 1 pg/ml to about 40 mg/ml or
(b) the tetracycline derivative is present at doses up to about 200 μg when the formulation is administered intravitreally or
(c) the tetracycline derivative is present at doses in the range of about 1 pg/ml to about 2 mg/ml when administered intraocularly or
(d) the tetracycline or a derivative thereof is selected from the group consisting of doxycycline, demeclocycline, minocycline, oxytetracycline, lymecycline, and a chemically modified tetracycline or
(e) the chemically modified tetracycline of (d) is selected from the group consisting of CMT-315, CMT-3, CMT-8, CMT-308 and 6-demethyl-6-deoxy-4-dedimethylamino tetracylcine (COL-3) or
(f) the tetracycline derivative is doxycycline or
(g) the concentration of doxycycline is between about 0.01 μg/ml to about 30 mg/ml or about 0.05 mg/ml to about 10 mg/ml or between about 1 mg/ml to about 20 mg/ml.

3. A formulation for the treatment of ocular neovascularization according to claim 1 wherein the formulation also includes a steroid at a concentration from about 0.1 mg/ml to about 40 mg/ml and optionally wherein
(a) the steroid is selected from the group consisting of triamcinolone, triamcinolone acetate, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate or
(b) the steroid is an 11-substituted-16α,17α-substituted methylenedioxy steroid or
(c) the steroid is 9-fluoro-11,21-ihydroxy-16,17-[1-methylethylidinebis(oxy)]pregna-1,4-diene-3,20-dione or
(d) the steroid concentration is about 0.1 mg/ml to about 40 mg/ml or
(e) the steroid concentration is about 1 mg/ml to about 20 mg/ml or
(f) the steroid concentration is about 20 mg/ml to about 30 mg/ml or
(g) the steroid concentration is about 20 mg/ml to about 40 mg/ml.

4. A formulation for the treatment of ocular neovascularization according to claim 1 wherein the formulation also includes an inhibitor of a metalloproteinase in a concentration and dose to reduce ocular neovascularization, wherein optionally
(a) the inhibitor of a metalloproteinase is selected from the group consisting of naturally occurring proteins such as TIMP-1 that specifically inhibit matrix metalloproteinases, and synthetic metalloproteinase inhibitors including Batimastat (BB-94) and marimastat (BB-2516) or
(b) the inhibitor of a metalloproteinase is selected from the group consisting of: inhibitors of collagenase I, II and III (MMP-1, MMP-8 and MMP-13), inhibitors of gelatinase A and B (MMP-2 and -9), inhibitors of stromelysin (MMP-3), inhibitors of matrilysin (MMP-7) and inhibitors of membrane type MMP (MMP-14).

5. An ocular pharmaceutically acceptable formulation comprising: an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization and
(a) a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml or
(b) a steroid such as triamcinolone acetonide at a concentration from about 0.1 mg/ml to about 40 mg/ml and a heparin or low molecular weight heparin in a concentration from about 0.01 pg/ml to about 100 mg/ml or
(c) a heparin or low molecular weight heparin in a concentration from about 0.01 pg/ml to about 100 mg/ml and a macrolide antibiotic such as ascomycin at a concentration from about 20 μg/ml to about 200 μg/ml or
(d) a heparin or low molecular weight heparin in a concentration from about 0.01 pg/ml to about 100 mg/ml and a tetracycline or a derivative thereof (including CMTs which inhibit MMP activity) such as doxycycline at a concentration from about 0.01 pg/ml to about 30 mg/ml or
(e) a heparin or low molecular weight heparin in a concentration from about 0.01 pg/ml to about 100 mg/ml and an inhibitor of a metalloproteinase in a concentration and dose to reduce ocular neovascularization or
(f) a steroid such as triamcinolone acetonide at a concentration from about 0.1 mg/ml to about 40 mg/ml and an inhibitor of a metalloproteinase in a concentration and dose to reduce ocular neovascularization or
(g) a steroid such as triamcinolone acetonide at a concentration from about 0.1 mg/ml to about 40 mg/ml and a macrolide antibiotic such as ascomycin at a concentration from about 20 μg/ml to about 200 μg/ml or
(h) a tetracycline or a derivative thereof (including CMTs which inhibit MMP activity) such as doxycycline at a concentration from about 0.01 pg/ml to about 30 mg/ml and an inhibitor of a metalloproteinase in a concentration and dose to reduce ocular neovascularization or
(i) a macrolide antibiotic such as ascomycin at a concentration from about 20 μg/ml to about 200 μg/ml and an inhibitor of a metalloproteinase in a concentration and dose to reduce ocular neovascularization or
(j) a tetracycline or a derivative thereof (including CMTs which inhibit MMP activity) such as doxycycline at a concentration from about 0.01 pg/ml to about 30 mg/ml, a steroid such as triamcinolone acetonide at a concentration from about 0.1 mg/ml to about 40 mg/ml and a heparin or low molecular weight heparin in a concentration from about 0.01 pg/ml to about 100 mg/ml or
(k) a tetracycline or a derivative thereof (including CMTs which inhibit MMP activity) such as doxycycline at a concentration from about 0.01 pg/ml to about 30 mg/ml, a steroid such as triamcinolone acetonide at a concentration from about 0.1 mg/ml to about 40 mg/ml and a macrolide antibiotic such as ascomycin at a concentration from about 20 μg/ml to about 200 μg/ml or
(l) a tetracycline or a derivative thereof (including CMTs which inhibit MMP activity) such as doxycycline at a concentration from about 0.01 pg/ml to about 30 mg/ml, a steroid such as triamcinolone acetonide at a concentration from about 0.1 mg/ml to about 40 mg/ml, a heparin or low molecular weight heparin in a concentration from about 0.01 pg/ml to about 100 mg/ml and a macrolide antibiotic such as ascomycin at a concentration from about 20 μg/ml to about 200 μg/ml.

6. A method for treating ocular neovascularization comprising the step of administering to a patient an antiprostaglandin in a concentration from about 0.001%^{w/v} to about 0.5%^{w/v}, characterized in that said antiprostaglandin is at a substantially neutral pH in a pharmaceutically acceptable form suitable for delivery to the eye to reduce ocular neovascularization.

7. A method for treating ocular neovascularization comprising the step of
(a) administering to a patient a formulation for sufficient time to treat the ocular neovascularization or
(b) administering to a patient a formulation following corneal surgery or
(c) administering to a patient a formulation to a patient in a cyclic tumor treatment regimen to reduce blood vessel growth and proliferation at a tumor site, and
wherein, the formulation is as defined in claim 1 for sufficient time to treat the ocular neovascularization and optionally wherein, the method includes the step of administering an anti-angiogenic agent designed to block the actions of VEGF on endothelial cells.

8. A method for treating ocular neovascularization comprising the step of
(a) administering to a patient a formulation for sufficient time to treat the ocular neovascularization or
(b) administering to a patient a formulation following corneal surgery or
(c) administering to a patient a formulation to a patient in a cyclic tumor treatment regimen to reduce blood vessel growth and proliferation at a tumor site, and
wherein, the formulation is as defined in claim 2 for sufficient time to treat the ocular neovascularization and optionally wherein, the method includes the step of administering an anti-angiogenic agent designed to block the actions of VEGF on endothelial cells.

9. A method for treating ocular neovascularization comprising the step of
(a) administering to a patient a formulation for sufficient time to treat the ocular neovascularization or
(b) administering to a patient a formulation following corneal surgery or
(c) administering to a patient a formulation to a patient in a cyclic tumor treatment regimen to reduce blood vessel growth and proliferation at a tumor site, and
wherein, the formulation is as defined in claim 3 for sufficient time to treat the ocular neovascularization and optionally wherein, the method includes the step of administering an anti-angiogenic agent designed to block the actions of VEGF on endothelial cells.

10. A method for treating ocular neovascularization comprising the step of
(a) administering to a patient a formulation for sufficient time to treat the ocular neovascularization or
(b) administering to a patient a formulation following corneal surgery or
(c) administering to a patient a formulation to a patient in a cyclic tumor treatment regimen to reduce blood vessel growth and proliferation at a tumor site, and
wherein, the formulation is as defined in claim 4 for sufficient time to treat the ocular neovascularization and optionally wherein, the method includes the step of administering an anti-angiogenic agent designed to block the actions of VEGF on endothelial cells.

11. A method for treating ocular neovascularization comprising the step of
(a) administering to a patient a formulation for sufficient time to treat the ocular neovascularization or
(b) administering to a patient a formulation following corneal surgery or
(c) administering to a patient a formulation to a patient in a cyclic tumor treatment regimen to reduce blood vessel growth and proliferation at a tumor site, and
wherein, the formulation is as defined in claim 5 for sufficient time to treat the ocular neovascularization and optionally wherein, the method includes the step of administering an anti-angiogenic agent designed to block the actions of VEGF on endothelial cells.

12. A method according to claim 7 wherein the anti-angiogenic agent is a rhuFab V2 or a humanized AMD-Fab or an anti-VEGF aptamer.

13. A method according to claim 8 wherein the anti-angiogenic agent is a rhuFab V2 or a humanized AMD-Fab or an anti-VEGF aptamer.

14. A method according to claim 9 wherein the anti-angiogenic agent is a rhuFab V2 or a humanized AMD-Fab or an anti-VEGF aptamer.

15. A method according to claim 10 wherein the anti-angiogenic agent is a rhuFab V2 or a humanized AMD-Fab or an anti-VEGF aptamer.

16. A method according to claim 11 wherein the anti-angiogenic agent is a rhuFab V2 or a humanized AMD-Fab or an anti-VEGF aptamer.
